(19) United States
(12) Patent Application Publication
(10) Pub. No.: US 2026/0258077 A1
(43) Pub. Date: Sep. 3, 2026

(54) PROTEIN FOLDING AGENT, COMPOUND, PROTEIN FOLDING METHOD, PHARMACEUTICAL COMPOSITION, AND AGENT FOR SUPPRESSING CYTOTOXICITY BY TRANSITION METAL

(71) Applicant: National University Corporation Tokyo University of Agriculture and Technology, Tokyo (JP)

(72) Inventors: Takahiro MURAOKA, Fuchu-shi (JP); Keita MORI, Fuchu-shi (JP)

(21) Appl. No.: 19/514,728

(22) PCT Filed: Aug. 28, 2024
(86) PCT No.: PCT/JP2024/030809

§ 371 Date: Feb. 26, 2026

(30) Foreign Application Priority Data
Aug. 31, 2023 (JP) 2023-140709
May 20, 2024 (JP) 2024-082219

(51) Int. Cl. C07K 1/113; A61K 31/395; C07D 255/02; C07D 257/02; C07D 513/08

(52) U.S. Cl. CPC C07K 1/1136; A61K 31/395; C07D 255/02; C07D 257/02; C07D 513/08

(57) ABSTRACT

A protein folding agent includes at least one or more compounds selected from the group consisting of (A) to (B): (A) a cyclic polyamine that contains one or more thiol groups or disulfide bonds and is bindable to a transition metal ion; (B) a compound in which two or more of the cyclic polyamines (A) are bonded through a disulfide bond between the thiol groups; (C) a complex of the cyclic polyamine (A) or the compound (B) with a transition metal ion; (D) a salt of the cyclic polyamine (A) or the compound (B); and (E) a solvate of the cyclic polyamine (A) or the compound (B).

The present application is a 35 U.S.C. § 371 filing of International Patent Application No. PCT/JP2024/030809, filed Aug. 28, 2024, which claims priority to JP2023-140709 filed on Aug. 31, 2023 and JP2024-082219 filed on May 20, 2024, the contents of which are incorporated herein by reference.

### TECHNICAL FIELD

The present invention relates to a protein folding agent, a compound, a protein folding method, a pharmaceutical composition, and an agent for suppressing cytotoxicity by a transition metal.

### BACKGROUND ART

Protein folding is a reaction in which a protein in a denatured state is refolded into a native structure having activity. Since the protein in a denatured state has a tendency to aggregate irreversibly, the efficiency of protein folding is generally low. Compounds that promote the protein folding are important as additives for efficiently obtaining active proteins in the production of industrially important proteins, such as pharmaceutical proteins. In addition, the protein in a denatured state is also implicated in folding diseases, such as neurodegenerative diseases. Accordingly, the compounds that promote folding are also important as drug candidates that can lead to prevention and/or treatment of folding diseases.

Proteins can be broadly classified into two types: proteins having intramolecular disulfide bonds and proteins not having intramolecular disulfide bonds. In vivo, folding proceeds through different mechanisms for each type. Folding of proteins that lack disulfide bonds is promoted by molecular chaperones typified by GroEL. Folding of proteins that have disulfide bonds is promoted by redox enzymes typified by protein disulfide isomerase (PDI) by inducing formation and exchange of disulfide bonds. The proteins that lack disulfide bonds are often present inside cells, that is, in the cytoplasm. In contrast, many membrane proteins and proteins secreted extracellularly have disulfide bonds. Many proteins that are useful as pharmaceuticals, such as antibodies and insulin, also have disulfide bonds.

In general, the proteins irreversibly aggregate in the presence of transition metal ions such as a nickel ion and a copper ion (Non-Patent Document 1). The property is considered to not only restrict protein synthesis conditions but also cause protein denaturation diseases (Non-Patent Documents 2 to 6).

As the compounds that promote folding of proteins having disulfide bonds, thiol compounds that promote redox reactions have been reported. For example, oxidized glutathione (GSSH) and reduced glutathione (GSH), nitrogen atom-containing thiol compounds (Patent Document 1), and the like have been reported.

### CITATION LIST

### Patent Document

Patent Document 1: Japanese Unexamined Patent Application, First Publication No. 2022-135954

### Non Patent Documents

Non-Patent Document 1: R. John et al., Probing the Role of Cu(II) Ions on Protein Aggregation Using Two Model Proteins. ACS Omega 2021, 6, 35559-35571.
Non-Patent Document 2: A. C. Kim et al., Metal Ion Effects on Aβ and Tau Aggregation. International Journal of Molecular Sciences 2018, 19 (1), 128.
Non-Patent Document 3: A. Prakash et al., Brain biometals and Alzheimer's disease—boon or bane? International Journal of Neuroscience 2017, 127, 99.
Non-Patent Document 4: P. B. Gabriel et al. Aggregation of biologically important peptides and proteins: inhibition or acceleration depending on protein and metal ion concentrations. RSC Advances 2020, 10, 215.
Non-Patent Document 5: M. Bisaglia, and L. Bubacco, Copper Ions and Parkinson's Disease: Why Is Homeostasis So Relevant? Biomolecules 2020, 10, 195.
Non-Patent Document 6: M. Tamas et al. Heavy Metals and Metalloids As a Cause for Protein Misfolding and Aggregation. Biomolecules 2014, 4, 252.

### SUMMARY OF INVENTION

### Technical Problem

With conventional folding agents such as glutathione (GSH/GSSH), there are no known agents capable of promoting protein folding in the presence of transition metal ions.

Accordingly, an object of the present invention is to provide a protein folding agent capable of promoting protein folding in the presence of transition metal ions; a compound usable in the protein folding agent; a protein folding method using the protein folding agent; a pharmaceutical composition containing the protein folding agent; and an agent for suppressing cytotoxicity by a transition metal.

### Solution to Problem

The present invention includes the following aspects.
[1] A protein folding agent including at least one or more compounds selected from the group consisting of (A) to (B): (A) a cyclic polyamine that contains one or more thiol groups or disulfide bonds and is bindable to a transition metal ion; (B) a compound in which two or more of the cyclic polyamines (A) are bonded through a disulfide bond between the thiol groups; (C) a complex of the cyclic polyamine (A) or the compound (B) with a transition metal ion; (D) a salt of the cyclic polyamine (A) or the compound (B); and (E) a solvate of the cyclic polyamine (A) or the compound (B).
[2] The protein folding agent according to [1], in which the cyclic polyamine (A) is a compound represented by General Formula (A).

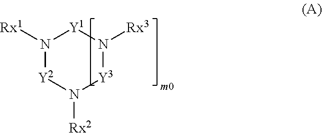

[In the formula, Rx¹ to Rx³ each independently represent a hydrogen atom or a monovalent organic group, provided that at least one of Rx¹ to Rx³ is a monovalent organic group containing one or more thiol groups, when Rx¹ to Rx³ contain two or more thiol groups in total, the thiol groups may be bonded to each other to form a disulfide bond, Y¹ to Y³ each independently represent an alkylene group having 1 to 5 carbon atoms, m0 represents an integer of 1 to 3, when m0 is an integer of 2 or more, two or more Rx³'s may be the same as or different from each other, and two or more Y³'s may be the same as or different from each other]
[3] The protein folding agent according to [2], in which the cyclic polyamine (A) is a compound represented by General Formula (A-1) or (A-2).

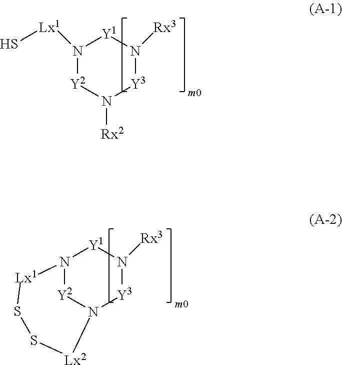

[In the formula, Rx² and Rx³ each independently represent a hydrogen atom or a monovalent organic group, Y¹ to Y³ each independently represent an alkylene group having 1 to 5 carbon atoms, Lx¹ and Lx² each independently represent an alkylene group having 1 to 10 carbon atoms, which may contain at least one selected from the group consisting of an oxygen atom and a nitrogen atom, m0 represents an integer of 1 to 3, and when m0 is an integer of 2 or more, two or more Rx³'s may be the same as or different from each other, and two or more Y³'s may be the same as or different from each other].
[4] The protein folding agent according to [3], in which the cyclic polyamine (A) is a compound represented by General Formula (A-1-1), and the compound (B) is a compound represented by General Formula (B-1-1).

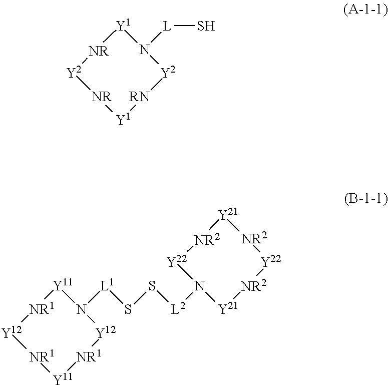

[In the formulae, Y¹, Y², Y¹¹, Y¹², Y²¹, and Y²² each independently represent an alkylene group having 1 to 5 carbon atoms, R, R¹, and R² each independently represent a hydrogen atom or an alkyl group having 1 to 3 carbon atoms, and L, L¹, and L² each independently represent an alkylene group having 1 to 10 carbon atoms, which may contain at least one selected from the group consisting of an oxygen atom and a nitrogen atom]
[5] The protein folding agent according to [4], in which the protein folding agent includes at least one compound selected from the group consisting of the compound (B), a metal complex of the compound (B), a salt of the compound (B), and a solvate of the compound (B).
[6] The protein folding agent according to [4], in which the compound (B) is a compound represented by Formula (B-1-1-1).

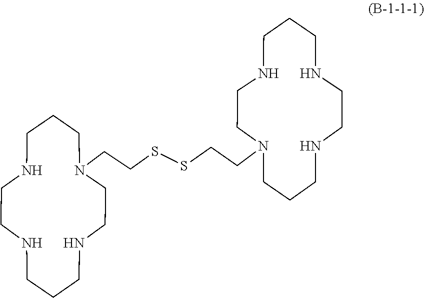 [SMILES: C1CNCCNCCCN(CCSSCCN2CCCNCCNCCCNCC2)CCNC1]

[7] The protein folding agent according to [4], in which the cyclic polyamine (A) is a compound represented by Formula (A-1-1-1), and the compound (B) is a compound represented by Formula (B-1-1-1).

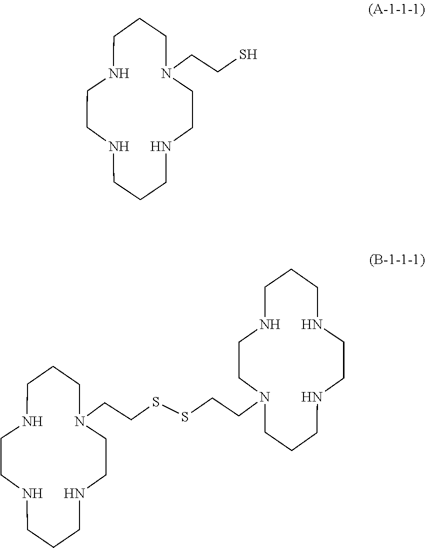 [SMILES: SCCN1CCCNCCNCCCNCC1 ; C1CNCCNCCCN(CCSSCCN2CCCNCCNCCCNCC2)CCNC1]

[8] The protein folding agent according to [3], in which the cyclic polyamine (A) is a compound represented by General Formula (A-1-2), and the compound (B) is a compound represented by General Formula (B-1-2).

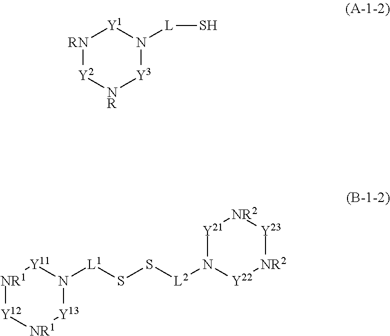

[In the formulae, Y¹, Y², Y³, Y¹¹, Y¹², Y¹³, Y²¹, Y²², and Y²³ each independently represent an alkylene group having 1 to 5 carbon atoms, R, R¹, and R² each independently represent a hydrogen atom or an alkyl group having 1 to 3 carbon atoms, and L, L¹, and L² each independently represent an alkylene group having 1 to 10 carbon atoms, which may contain at least one selected from the group consisting of an oxygen atom and a nitrogen atom]

[9] The protein folding agent according to [3], in which the cyclic polyamine (A) is a compound represented by Formula (A-1-2-1), and the compound (B) is a compound represented by Formula (B-1-2-1).

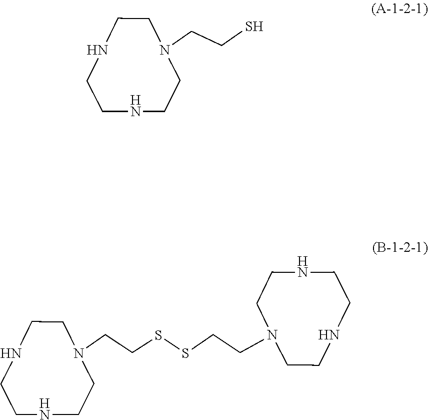 [SMILES: SCCN1CCNCCNCC1 ; C1CNCCN(CCSSCCN2CCNCCNCC2)CCN1]

[10] The protein folding agent according to any one of [1] to [9], in which the protein folding agent includes at least one compound selected from the group consisting of a cyclic polyamine (A), a metal complex of the cyclic polyamine (A), a salt of the cyclic polyamine (A), and a solvate of the cyclic polyamine (A), and at least one compound selected from the group consisting of the compound (B), a metal complex of the compound (B), a salt of the compound (B), and a solvate of the compound (B).
[11] The protein folding agent according to [3], in which the cyclic polyamine (A) is a compound represented by Formula (A-2-1).

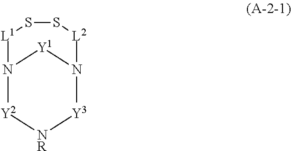

[In the formula, Y¹ to Y³ each independently represent an alkylene group having 1 to 5 carbon atoms, R represents a hydrogen atom or an alkyl group having 1 to 3 carbon atoms, and L¹ and L² each independently represent an alkylene group having 1 to 10 carbon atoms, which may contain at least one selected from the group consisting of an oxygen atom and a nitrogen atom]
[12] The protein folding agent according to [11], in which the cyclic polyamine (A) is a compound represented by Formula (A-2-1-1).

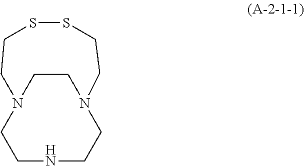 [SMILES: C1CN2CCSSCCN(CCN1)CC2]

[13] The protein folding agent according to any one of [1] to [12], in which the protein folding agent is used for folding a protein in a presence of a transition metal ion.
[14] The protein folding agent according to [13], in which the protein folding agent is used for folding a protein containing a transition metal ion.
[15] The protein folding agent according to or [14], in which the transition metal ion is at least one selected from the group consisting of a copper ion, a nickel ion, an iron ion, a manganese ion, a zinc ion, and a molybdenum ion.
[16] A compound represented by General Formula (B-1), (A-1-2), or (A-2).

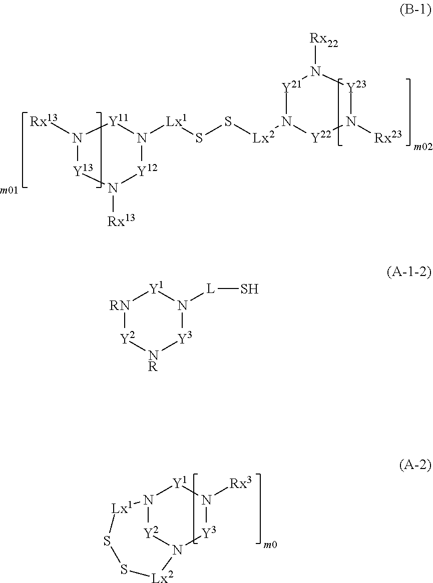

[In General Formula (B-1), Rx¹², Rx¹³, Rx²², and Rx²³ each independently represent a hydrogen atom or a monovalent organic group, Y¹¹, Y¹², Y¹³, Y²¹, Y²², and Y²³ each independently represent an alkylene group having 1 to 5 carbon atoms, Lx¹ and Lx² each independently represent an alkylene group having 1 to 10 carbon atoms, which may contain at least one selected from the group consisting of an oxygen atom and a nitrogen atom, m01 and m02 each independently represent an integer of 1 to 3, when m01 is an integer of 2 or more, two or more Rx¹³'s may be the same as or different from each other, and two or more Y¹³'s may be the same as or different from each other, and when m02 is an integer of 2 or more, two or more Rx²³'s may be the same as or different from each other, and two or more Y²³'s may be the same as or different from each other;
in General Formula (A-1-2), Y¹ to Y³ each independently represent an alkylene group having 1 to 5 carbon atoms, R's each independently represent a hydrogen atom or an alkyl group having 1 to 3 carbon atoms, and L represents an alkylene group having 1 to 10 carbon atoms, which may contain at least one selected from the group consisting of an oxygen atom and a nitrogen atom;
in General Formula (A-2), Rx³ represents a hydrogen atom or a monovalent organic group, Y¹ to Y³ each independently represent an alkylene group having 1 to 5 carbon atoms, Lx¹ and Lx² each independently represent an alkylene group having 1 to carbon atoms, which may contain at least one selected from the group consisting of an oxygen atom and a nitrogen atom, m0 represents an integer of 1 to 3, and when m0 is an integer of 2 or more, two or more Rx³'s may be the same as or different from each other, and two or more Y³'s may be the same as or different from each other]
[17] The compound according to [16], in which the compound is a compound represented by Formula (B-1-1-1), (A-1-2-1), (B-1-2-1), or (A-2-1-1).

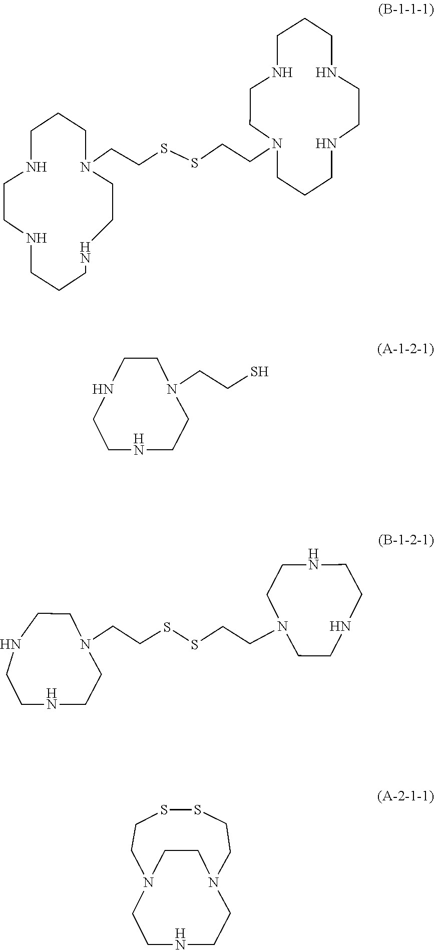 [SMILES: C1CNCCNCCCN(CCSSCCN2CCCNCCNCCCNCC2)CCNC1 ; SCCN1CCNCCNCC1 ; C1CNCCN(CCSSCCN2CCNCCNCC2)CCN1 ; C1CN2CCSSCCN(CCN1)CC2]

[18] A protein folding method including: a step of incubating a protein in a presence of the protein folding agent according to any one of [1] to [12].
[19] The protein folding method according to [18], in which the protein is an unfolded protein or a misfolded protein.
[20] The protein folding method according to or [19], in which the incubation is performed in a presence of a transition metal ion.
[21] The protein folding method according to [20], in which a folded protein after the incubating step contains the transition metal ion.
[22] The protein folding method according to [20], in which the transition metal ion is at least one selected from the group consisting of a copper ion, a nickel ion, an iron ion, a manganese ion, a zinc ion, and a molybdenum ion.
[23] A pharmaceutical composition containing: the protein folding agent according to any one of [1] to [12]; and a pharmaceutically acceptable carrier.
[24] The pharmaceutical composition according to [23], in which the pharmaceutical composition is used for treatment or prevention of a disease caused by aggregation of proteins.
[25] The pharmaceutical composition according to [24], in which the disease caused by aggregation of proteins is a folding disease.
[26] The pharmaceutical composition according to [25], in which the folding disease is at least one selected from the group consisting of amyotrophic lateral sclerosis (ALS), Parkinson's disease, Alzheimer's disease, dementia with Lewy bodies, spinocerebellar degeneration, frontotemporal dementia, Pick's disease, Creutzfeldt-Jakob disease, familial amyloid polyneuropathy, and type 2 diabetes.
[27] An agent for suppressing cytotoxicity by a transition metal, containing at least one or more compounds selected from the group consisting of (A) to (B): (A) a cyclic polyamine that contains one or more thiol groups or disulfide bonds and is bindable to a transition metal ion; (B) a compound in which two or more of the cyclic polyamines (A) are bonded through a disulfide bond between the thiol groups; (C) a complex of the cyclic polyamine (A) or the compound (B) with a transition metal ion; (D) a salt of the cyclic polyamine (A) or the compound (B); and (E) a solvate of the cyclic polyamine (A) or the compound (B).

### Advantageous Effects of Invention

According to the present invention, there are provided a protein folding agent capable of promoting protein folding in the presence of transition metal ions; a compound usable in the protein folding agent; a protein folding method using the protein folding agent; and a pharmaceutical composition containing the protein folding agent.

### BRIEF DESCRIPTION OF DRAWINGS

FIG. 1 Results of 1H NMR measurements for confirming the structure of a compound 5 that was an intermediate of compounds (A-1-1-1) and (B-1-1-1) synthesized in Experimental Example 1.

FIG. 2 Results of MALDI-TOF mass spectrometric spectrum measurements for confirming the structure of the compound 5 that was an intermediate of compounds (A-1-1-1) and (B-1-1-1) synthesized in Experimental Example 1.

FIG. 3 A result of 1H NMR measurement for confirming the structure of the compound (B-1-1-1) synthesized in Experimental Example 1.

FIG. 4 A result of MALDI-TOF mass spectrometric spectrum measurement for confirming the structure of the compound (B-1-1-1) synthesized in Experimental Example 1.

FIG. 5 A result of 1H NMR measurement for confirming the structure of the compound (A-1-1-1) synthesized in Experimental Example 1.

FIG. 6 A result of MALDI-TOF mass spectrometric spectrum measurement for confirming the structure of the compound (A-1-1-1) synthesized in Experimental Example 1.

FIG. 7 Results of a folding test of reduced and denatured Bovine Pancreatic Trypsin Inhibitor (BPTI) using oxidized glutathione (GSSG) and reduced glutathione (GSH) in Experimental Example 1.

FIG. 8 Results of a folding test of reduced and denatured BPTI using the compound (A-1-1-1) and the compound (B-1-1-1) in Experimental Example 1.

FIG. 9 Results of a folding test of reduced and denatured BPTI in the presence of nickel (II) ions (Ni²⁺), using the compound (A-1-1-1) and the compound (B-1-1-1) in Experimental Example 1.

FIG. 10 Results of a folding test of reduced and denatured BPTI in the presence of copper (II) ions (Cu²⁺), using the compound (A-1-1-1) and the compound (B-1-1-1) in Experimental Example 1.

FIG. 11 Results of a folding test of reduced and denatured BPTI under various conditions using GSSG and GSH, or the compound (A-1-1-1) and the compound (B-1-1-1) in Experimental Example 1.

FIG. 12 Results of a folding test of reduced and denatured BPTI in the presence of zinc (II) ions (Zn²⁺), using GSSG and GSH, or the compound (A-1-1-1) and the compound (B-1-1-1) in Experimental Example 1.

FIG. 13 Results of a cytotoxicity evaluation test of a mixture of the compound (A-1-1-1) and the compound (B-1-1-1) in Experimental Example 1.

FIG. 14 Results of a test for evaluating a suppressing effect of a mixture of the compound (A-1-1-1) and the compound (B-1-1-1) on cytotoxicity of copper (II) ions (Cu²⁺) in Experimental Example 1.

FIG. 15 Results of a test for evaluating a suppressing effect of the compound (B-1-1-1) on cytotoxicity of copper (II) ions (Cu²⁺) in Experimental Example 1.

FIG. 16 Results of 1H NMR measurements for confirming the structure of a compound 8 that was an intermediate of compounds (A-1-2-1) and (B-1-2-1) synthesized in Experimental Example 2.

FIG. 17 Results of MALDI-TOF mass spectrometric spectrum measurements for confirming the structure of the compound 8 that was an intermediate of compounds (A-1-2-1) and (B-1-2-1) synthesized in Experimental Example 2.

FIG. 18 A result of 1H NMR measurement for confirming the structure of the compound (B-1-2-1) synthesized in Experimental Example 2.

FIG. 19 A result of MALDI-TOF mass spectrometric spectrum measurement for confirming the structure of the compound (B-1-2-1) synthesized in Experimental Example 2.

FIG. 20 A result of 1H NMR measurement for confirming the structure of the compound (A-1-2-1) synthesized in Experimental Example 2.

FIG. 21 A result of MALDI-TOF mass spectrometric spectrum measurement for confirming the structure of the compound (A-1-2-1) synthesized in Experimental Example 2.

FIG. 22 Results of a folding test of reduced and denatured BPTI using GSSG and GSH, or the compound (A-1-2-1) and the compound (B-1-2-1) in Experimental Example 2.

FIG. 23 Results of a folding test of reduced and denatured BPTI in the presence of zinc (II) ions (Zn²⁺), using the compound (B-1-2-1) in Experimental Example 2.

FIG. 24 Results of a folding test of reduced and denatured BPTI in the presence of zinc (II) ions (Zn²⁺), using GSSG and GSH, or the compound (A-1-2-1) and the compound (B-1-2-1) in Experimental Example 2.

FIG. 25 Results of a folding test of reduced and denatured BPTI in the presence of zinc (II) ions (Zn²⁺), using GSSG or the compound (B-1-2-1) in Experimental Example 2.

FIG. 26 A result of 1H NMR measurement for confirming the structure of the compound (B-2-1-1) synthesized in Experimental Example 3.

FIG. 27 A result of MALDI-TOF mass spectrometric spectrum measurement for confirming the structure of the compound (B-2-1-1) synthesized in Experimental Example 3.

FIG. 28 Results of a folding test of reduced and denatured BPTI using GSSG or the compound (B-2-1-1) in Experimental Example 3.

FIG. 29 Results of a folding test of reduced and denatured BPTI in the presence of copper (II) ions (Cu²⁺), using the compound (B-2-1-1) in Experimental Example 3.

FIG. 30 Results of a folding test of reduced and denatured BPTI in the presence of nickel (II) ions (Ni²⁺), using the compound (B-2-1-1) in Experimental Example 3.

FIG. 31 Results of a folding test of reduced and denatured BPTI using a catalytic amount of the compound (B-2-1-1) in Experimental Example 3.

FIG. 32 Results of a folding test of reduced and denatured BPTI in the presence of nickel (II) ions (Ni²⁺), using a catalytic amount of the compound (B-2-1-1) in Experimental Example 3.

FIG. 33 Results of a folding test of reduced and denatured BPTI in the presence of copper (II) ions (Cu²⁺), using a catalytic amount of the compound (B-2-1-1) in Experimental Example 3.

FIG. 34 Results of a folding test of reduced and denatured RNAase A in the presence of copper (II) ions (Cu²⁺), using GSSG and GSH, or the compound (A-1-1-1) and the compound (B-1-1-1) in Experimental Example 4.

### DESCRIPTION OF EMBODIMENTS

A numerical range represented by “to” refers to a range including numerical values denoted before and after “to” as a lower limit value and an upper limit value.

The term “includes” (comprises) means that constituent elements other than the target constituent elements may be included. The term “consists of” means that constituent elements other than the target constituent elements are not included. The term “consist essentially of” means that elements other than specified elements are not included in an aspect that exhibits a special function (such as an aspect that completely loses the effect of the invention). In the present specification, the term “includes” (comprises) encompasses the meanings of “consists of” and “consists essentially of”.

### [Protein Folding Agent]

A first aspect of the present disclosure is a protein folding agent. The protein folding agent according to the present aspect includes at least one or more compounds selected from the group consisting of (A) to (B).
(A) a cyclic polyamine that contains one or more thiol groups or disulfide bonds and is bindable to a transition metal ion (hereinafter, also referred to as “compound (A)”);
(B) a compound in which two or more of the cyclic polyamines (A) are bonded through a disulfide bond between the thiol groups (hereinafter, also referred to as “compound (B)”);
(C) a complex of the cyclic polyamine (A) or the compound (B) with a transition metal ion (hereinafter, also referred to as “compound (C)”);
(D) a salt of the cyclic polyamine (A) or the compound (B) (hereinafter, also referred to as “compound (D)”); and
(E) a solvate of the cyclic polyamine (A) or the compound (B) (hereinafter, also referred to as “compound (E)”)

### <Compound (A)>

In the present specification, the term “cyclic polyamine” refers to a compound containing an aliphatic heterocycle having two or more nitrogen atoms in a ring structure. The cyclic polyamine preferably contains, in the ring structure, two or more amino groups selected from the group consisting of a secondary amino group and a tertiary amino group. The number of thiol groups or disulfide bonds in the compound (A) is, for example, 1 to 5, preferably 1 to 3, more preferably 1 or 2, and still more preferably 1.

The compound (A) can bind to a transition metal ion by a coordination bond. The transition metal is an element located between Group 3 and Group 12 in the periodic table. Examples of the transition metal ion include, but are not limited to, a copper ion, a nickel ion, an iron ion, a manganese ion, a zinc ion, and a molybdenum ion.

Examples of the compound (A) include a compound represented by General Formula (A).

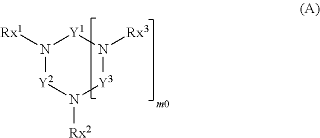

[In the formula, Rx¹ to Rx³ each independently represent a hydrogen atom or a monovalent organic group, provided that at least one of Rx¹ to Rx³ is a monovalent organic group containing one or more thiol groups, when Rx¹ to Rx³ contain two or more thiol groups in total, the thiol groups may be bonded to each other to form a disulfide bond, Y¹ to Y³ each independently represent an alkylene group having 1 to 5 carbon atoms, m0 represents an integer of 1 to 3, when m0 is an integer of 2 or more, two or more Rx³'s may be the same as or different from each other, and two or more Y³'s may be the same as or different from each other]

In Formula (A), examples of the monovalent organic group for Rx¹ to Rx³ include a monovalent hydrocarbon group that may have a substituent. The above-described monovalent hydrocarbon group may be an aliphatic hydrocarbon group or an aromatic hydrocarbon group. Examples of the monovalent organic group for Rx¹ to Rx³ include monovalent organic groups having 1 to 20 carbon atoms, preferably 1 to 16 carbon atoms, more preferably 1 to 10 carbon atoms, and still more preferably 1 to 6 carbon atoms.

Examples of the aliphatic hydrocarbon group for Rx 1 to Rx³ include aliphatic hydrocarbon groups having 1 to 10 carbon atoms, preferably 1 to 6 carbon atoms, more preferably 1 to 3 carbon atoms, still more preferably 1 or 2 carbon atoms, and particularly preferably 1 carbon atom. The aliphatic hydrocarbon group may be saturated or unsaturated, but is preferably saturated. The aliphatic hydrocarbon group may be linear or branched, or may include a ring structure. The aliphatic hydrocarbon group is preferably linear or branched, and more preferably linear.

The aliphatic hydrocarbon group is preferably a linear or branched alkyl group. Examples of the linear alkyl group include linear alkyl groups having 1 to 10 carbon atoms, preferably 1 to 6 carbon atoms, more preferably 1 to 3 carbon atoms, still more preferably 1 or 2 carbon atoms, and particularly preferably 1 carbon atom. Examples of the branched alkyl group include branched alkyl groups having 3 to 10 carbon atoms, preferably 3 to 6 carbon atoms, more preferably 3 or 4 carbon atoms, and still more preferably 3 carbon atoms.

The aromatic hydrocarbon group for Rx¹ to Rx³ is a hydrocarbon group containing one or more aromatic rings. Examples of the aromatic hydrocarbon group for Rx¹ to Rx³ include aromatic hydrocarbon groups having 1 to 20 carbon atoms, preferably 1 to 16 carbon atoms, more preferably 1 to 10 carbon atoms, and still more preferably 1 to 6 carbon atoms. The aromatic ring contained in the above-described aromatic hydrocarbon group may be monocyclic or polycyclic, but is preferably monocyclic. The above-described aromatic ring may be an aromatic hydrocarbon ring or an aromatic heterocyclic ring. Examples of the above-described aromatic hydrocarbon ring include a benzene ring and a naphthalene ring. Examples of the above-described aromatic heterocyclic ring include nitrogen-containing aromatic heterocyclic rings such as pyrrole, sulfur-containing aromatic heterocyclic rings such as thiophene, and oxygen-containing aromatic heterocyclic rings such as furan.

Examples of the aromatic hydrocarbon group for Rx¹ to Rx³ include a group obtained by removing one hydrogen atom from an aromatic ring, a group in which an aromatic ring is substituted at one carbon atom with an alkylene group, and a group in which an aromatic ring intervenes in the middle of an alkylene group.

The monovalent hydrocarbon group for Rx¹ to Rx³ may have a substituent. Examples of the above-described substituent include a thiol group, a hydroxy group, an amino group, a halogen atom, an alkoxy group, and an acyl group. The above-described substituent may be a divalent group that substitutes a methylene group. Examples of such a substituent include a carbonyl group, an ether bond (—O—), an ester bond (—COO—), and an amide bond (—NHCO—).

At least one of Rx¹ to Rx³ is a monovalent organic group containing a thiol group (hereinafter, also referred to as “thiol group-containing group”). The number of thiol group-containing groups includes, for example, 1 to 5, preferably 1 to 3, and more preferably 1 or 2. When there are two or more thiol group-containing groups, two thiol group-containing groups may bond to each other to form a disulfide bond. The number of thiol groups contained in one thiol group-containing group is preferably 1 to 5, more preferably 1 to 3, still more preferably 1 or 2, and even more preferably 1.

In Formula (A), the alkylene group having 1 to 5 carbon atoms for Y¹ to Y³ may be linear or branched, but is preferably linear. The above-described alkylene group preferably has 1 to 4 carbon atoms, and more preferably 2 or 3 carbon atoms.

In Formula (A), m0 is preferably 1 or 2.

Examples of the compound (A) include a compound represented by General Formula (A-1) (hereinafter, also referred to as “compound (A-1)”) and a compound represented by General Formula (A-2) (hereinafter, also referred to as “compound (A-2)”).

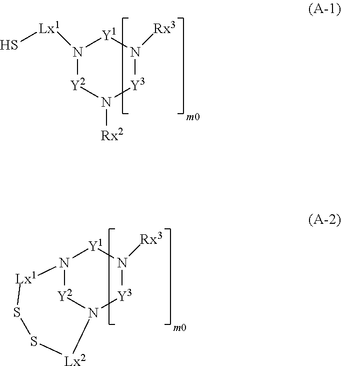

[in the formula, Rx² and Rx³ each independently represent a hydrogen atom or a monovalent organic group, Y¹ to Y³ each independently represent an alkylene group having 1 to 5 carbon atoms, Lx¹ and Lx² each independently represent an alkylene group having 1 to 10 carbon atoms, which may contain at least one selected from the group consisting of an oxygen atom and a nitrogen atom, m0 represents an integer of 1 to 3, and when m0 is an integer of 2 or more, two or more Rx³'s may be the same as or different from each other, and two or more Y³'s may be the same as or different from each other]

In Formulae (A-1) and (A-2), Rx², Rx³, Y¹ to Y³, and m0 are the same as Rx², Rx³, Y¹ to Y³, and m0 in Formula (A).

In Formulae (A-1) and (A-2), the alkylene group having 1 to 10 carbon atoms for Lx¹ and Lx² may be linear or branched, but is preferably linear. The alkylene group for Lx¹ and Lx² preferably has 1 to 8 carbon atoms, more preferably 1 to 6 carbon atoms, still more preferably 1 to 4 carbon atoms, even more preferably 1 to 3 carbon atoms, and particularly preferably 2 carbon atoms.

The alkylene group for Lx¹ and Lx² may contain at least one selected from the group consisting of an oxygen atom and a nitrogen atom. Examples of the group containing at least one selected from the group consisting of an oxygen atom and a nitrogen atom, which may be contained in the alkylene group for Lx¹ and Lx², include an ether bond (—O—), a group represented by —NR^{L}— (R^{L} represents a hydrogen atom or an alkyl group having 1 to 3 carbon atoms), an ester bond (—COO—), and an amide bond (—CONH—). R^{L} is preferably a hydrogen atom, a methyl group, or an ethyl group, and more preferably a hydrogen atom or a methyl group. In Lx¹ and Lx², a part of methylene groups (—CH₂—) constituting the alkylene group may be substituted with at least one selected from the group consisting of —O—, —NRL-, —COO—, and —CONH—.

Specific examples of the alkylene group for Lx¹ and Lx² include a methylene group, an ethylene group, a n-propylene group, an isopropylene group, a n-butylene group, an isobutylene group, a tert-butylene group, a n-pentylene group, an isopentylene group, a neopentylene group, a hexylene group, a heptylene group, an octylene group, a nonylene group, and a decylene group. A part of methylene groups (—CH₂—) constituting these alkylene groups may be substituted with a group containing at least one selected from the group consisting of an oxygen atom and a nitrogen atom (for example, —O—, —NR^{L}—, —COO—, and/or —CONH—).

Lx¹ and Lx² may be polyoxyalkylene groups. Specific examples of the polyoxyalkylene group include a polyoxyethylene group and a polyoxypropylene group.

Among the above, Lx¹ and Lx² are preferably an alkylene group having 1 to 6 carbon atoms or a polyoxyalkylene group having 1 to 6 carbon atoms; more preferably an alkylene group having 1 to 6 carbon atoms, a polyoxyethylene group having 1 to 6 carbon atoms, or a polyoxypropylene group having 1 to 6 carbon atoms; still more preferably an ethylene group or a propylene group; and particularly preferably an ethylene group.

Examples of the compound (A-1) include a compound represented by General Formula (A-1-1) (hereinafter, also referred to as “compound (A-1-1)”) and a compound represented by General Formula (A-1-2) (hereinafter, also referred to as “compound (A-1-2)”).

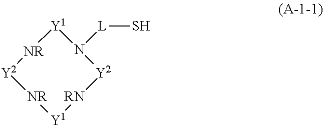

[in the formula, Y¹ and Y² each independently represent an alkylene group having 1 to 5 carbon atoms, R's each independently represent a hydrogen atom or an alkyl group having 1 to 3 carbon atoms, and L represents an alkylene group having 1 to carbon atoms, which may contain at least one selected from the group consisting of an oxygen atom and a nitrogen atom]

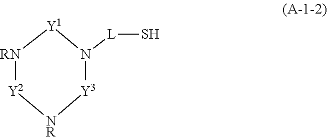

[in the formula, Y¹ to Y³ each independently represent an alkylene group having 1 to 5 carbon atoms, R's each independently represent a hydrogen atom or an alkyl group having 1 to 3 carbon atoms, and L represents an alkylene group having 1 to 10 carbon atoms, which may contain at least one selected from the group consisting of an oxygen atom and a nitrogen atom]

In Formula (A-1-1), Y¹ and Y² are the same as Y¹ and Y² in Formula (A-1) described above. Y¹ is preferably a linear alkylene group having 2 or 3 carbon atoms, and more preferably a propylene group. Two Y¹'s may be the same as or different from each other, but are preferably the same. Y² is preferably a linear alkylene group having 2 or 3 carbon atoms, and more preferably an ethylene group. Two Y²'s may be the same as or different from each other, but are preferably the same.

In Formula (A-1-2), Y¹ to Y³ are the same as Y¹ to Y³ in Formula (A-1) described above. Y¹ to Y³ are preferably linear alkylene groups having 2 or 3 carbon atoms, and more preferably ethylene groups. Y¹ to Y³ may be the same as or different from each other, but are preferably the same.

In Formulae (A-1-1) and (A-1-2), the alkyl group having 1 to 3 carbon atoms for R may be linear or branched, but is preferably linear. The linear alkyl group preferably has 1 or 2 carbon atoms, and more preferably 1 carbon atom. When the alkyl group for

R has 3 carbon atoms, it may be a branched alkyl group. Examples of the alkyl group for R include a methyl group, an ethyl group, a n-propyl group, and an isopropyl group, preferably a methyl group or an ethyl group, and more preferably a methyl group.

In Formulae (A-1-1) and (A-1-2), three R's may be the same as or different from each other, but are preferably the same.

R is preferably a hydrogen atom.

In Formulae (A-1-1) and (A-1-2), examples of L include the same as those for Lx¹ in (A-1) described above. L is preferably an alkylene group having 1 to 6 carbon atoms or a polyoxyalkylene group having 1 to 6 carbon atoms; more preferably an alkylene group having 1 to 6 carbon atoms, a polyoxyethylene group having 1 to 6 carbon atoms, or a polyoxypropylene group having 1 to 6 carbon atoms; still more preferably an ethylene group or a propylene group; and particularly preferably an ethylene group.

Examples of the compound (A-1-1) include a compound represented by General Formula (A-1-1a) (hereinafter, also referred to as “compound (A-1-1a)”).

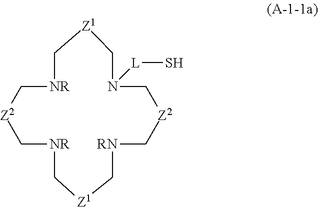

[in the formula, Z¹ and Z² each independently represent a single bond or a methylene group, R's each independently represent a hydrogen atom or an alkyl group having 1 to 3 carbon atoms, and L's each independently represent an alkylene group having 1 to 10 carbon atoms, which may contain at least one selected from the group consisting of an oxygen atom and a nitrogen atom]

In Formula (A-1-1a), two Z¹'s may be the same or different, but are preferably the same. Z¹ is preferably a methylene group.

In Formula (A-1-1a), two Z²'s may be the same or different, but are preferably the same. Z² is preferably a single bond.

In Formula (A-1-1a), the alkyl group for R preferably has 1 or 2 carbon atoms, and more preferably 1 carbon atom. When the alkyl group for R has 3 carbon atoms, it may be a branched alkyl group. Examples of the alkyl group for R include a methyl group, an ethyl group, a n-propyl group, and an isopropyl group, preferably a methyl group or an ethyl group, and more preferably a methyl group.

In Formula (A-1-1a), three R's may be the same as or different from each other, but are preferably the same.

R is preferably a hydrogen atom.

In Formula (A-1-1a), the alkylene group for L may be linear or branched, but is preferably linear. The alkylene group for L preferably has 1 to 8 carbon atoms, more preferably 1 to 6 carbon atoms, still more preferably 1 to 4 carbon atoms, even more preferably 1 to 3 carbon atoms, and particularly preferably 2 carbon atoms.

The alkylene group for L may contain at least one selected from the group consisting of an oxygen atom and a nitrogen atom. Examples of the group containing at least one selected from the group consisting of an oxygen atom and a nitrogen atom, which may be contained in the alkylene group for L, include an ether bond (—O—), a group represented by —NR^{L}—(R^{L} represents a hydrogen atom or an alkyl group having 1 to 3 carbon atoms), an ester bond (—COO—), and an amide bond (—CONH—). R^{L} is preferably a hydrogen atom, a methyl group, or an ethyl group, and more preferably a hydrogen atom or a methyl group. In L, a part of methylene groups (—CH₂—) constituting the alkylene group may be substituted with at least one selected from the group consisting of —O—, —NRL-, —COO—, and —CONH—.

Specific examples of the alkylene group for L include a methylene group, an ethylene group, a n-propylene group, an isopropylene group, a n-butylene group, an isobutylene group, a tert-butylene group, a n-pentylene group, an isopentylene group, a neopentylene group, a hexylene group, a heptylene group, an octylene group, a nonylene group, and a decylene group. A part of methylene groups (—CH₂—) constituting these alkylene groups may be substituted with a group containing at least one selected from the group consisting of an oxygen atom and a nitrogen atom (for example, —O—, —NR^{L}—, —COO—, and/or —CONH—).

L may be a polyoxyalkylene group. Specific examples of the polyoxyalkylene group include a polyoxyethylene group and a polyoxypropylene group.

Among the above, L is preferably an alkylene group having 1 to 6 carbon atoms or a polyoxyalkylene group having 1 to 6 carbon atoms; more preferably an alkylene group having 1 to 6 carbon atoms, a polyoxyethylene group having 1 to 6 carbon atoms, or a polyoxypropylene group having 1 to 6 carbon atoms; still more preferably an ethylene group or a propylene group; and particularly preferably an ethylene group.

The compound (A-1-1a) is preferably a compound represented by Formula (A-1-1b) (hereinafter, also referred to as “compound (A-1-1b)”).

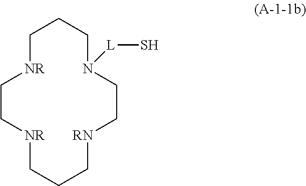

[in the formula, R's each independently represent a hydrogen atom or an alkyl group having 1 to 3 carbon atoms, and L represents an alkylene group having 1 to 10 carbon atoms, which may contain at least one selected from the group consisting of an oxygen atom and a nitrogen atom]

R in Formula (A-1-1b) is the same as R in Formula (A-1-1) described above. R is preferably a hydrogen atom or a methyl group, and more preferably a hydrogen atom.

L in Formula (A-1-1b) is the same as L in Formula (A-1-1) described above. L is preferably an alkylene group having 1 to 6 carbon atoms or a polyoxyalkylene group having 1 to 6 carbon atoms; more preferably an alkylene group having 1 to 6 carbon atoms, a polyoxyethylene group having 1 to 6 carbon atoms, or a polyoxypropylene group having 1 to 6 carbon atoms; still more preferably an ethylene group or a propylene group; and particularly preferably an ethylene group.

Specific examples of the compound (A-1-1b) include a compound represented by Formula (A-1-1-1) (hereinafter, also referred to as “compound (A-1-1-1)”).

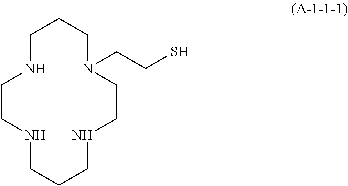 [SMILES: SCCN1CCCNCCNCCCNCC1]

Specific examples of the compound (A-1-2) include a compound represented by Formula (A-1-2-1) (hereinafter, also referred to as “compound (A-1-2-1)”).

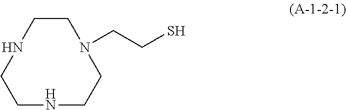 [SMILES: SCCN1CCNCCNCC1]

Examples of the compound (A-2) include a compound represented by General Formula (A-2-1) (hereinafter, also referred to as “compound (A-2-1)”).

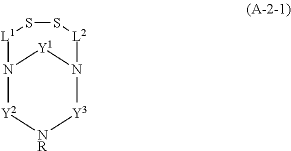

[in the formula, Y¹ to Y³ each independently represent an alkylene group having 1 to 5 carbon atoms, R represents a hydrogen atom or an alkyl group having 1 to 3 carbon atoms, and L¹ and L² each independently represent an alkylene group having 1 to 10 carbon atoms, which may contain at least one selected from the group consisting of an oxygen atom and a nitrogen atom]

In Formula (A-2-1), Y¹ to Y³ and R are the same as Y¹ to Y³ and R in Formula (A-1-2) described above.

In Formula (A-2-1), examples of L¹ and L² include the same ones as those of Lx¹ in (A-1) described above. L¹ and L² are each preferably an alkylene group having 1 to 6 carbon atoms or a polyoxyalkylene group having 1 to 6 carbon atoms; more preferably an alkylene group having 1 to 6 carbon atoms, a polyoxyethylene group having 1 to 6 carbon atoms, or a polyoxypropylene group having 1 to 6 carbon atoms; still more preferably an ethylene group or a propylene group; and particularly preferably an ethylene group.

L¹ and L² may be the same as or different from each other, but are preferably the same.

Specific examples of the compound (A-2-1) include a compound represented by Formula (A-2-1-1) (hereinafter, also referred to as “compound (A-2-1-1)”).

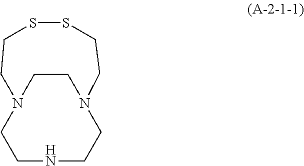 [SMILES: C1CN2CCSSCCN(CCN1)CC2]

### <Compound (B)>

The compound (B) is a compound in which two or more compounds (A) are bonded through a disulfide bond between thiol groups. The compound (B) is, for example, a compound in which two molecules of the compound (A) are bonded through a disulfide bond (—S—S—). Two or more compounds (A) bonded through a disulfide bond may be the same compound or different compounds, but are preferably the same compound.

Examples of the compound (B) include a compound in which two molecules of the above-described compound (A-1) are bonded through a disulfide bond. Two molecules of the compound (A-1) bonded through a disulfide bond may be the same compound or different compounds, but are preferably the same compound.

Examples of the compound (B) include a compound represented by General Formula (B-1) (hereinafter, also referred to as “compound (B-1)”). The compound (B-1) is a compound in which two molecules of the compound (A-1) are bonded through a disulfide bond.

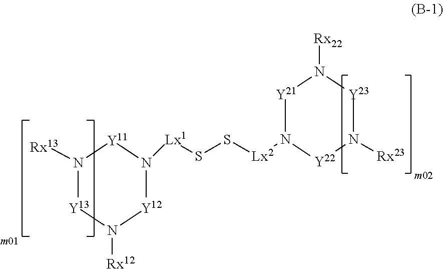

[in the formula, Rx¹², Rx¹³, Rx²² and Rx²³ each independently represent a hydrogen atom or a monovalent organic group, Y¹ to Y³ each independently represent an alkylene group having 1 to 5 carbon atoms, Lx¹ and Lx² each independently represent an alkylene group having 1 to 10 carbon atoms, which may contain at least one selected from the group consisting of an oxygen atom and a nitrogen atom, m01 and m02 each independently represent an integer of 1 to 3, when m01 is an integer of 2 or more, two or more Rx¹³'s may be the same as or different from each other, and two or more Y¹³'s may be the same as or different from each other, and when m02 is an integer of 2 or more, two or more Rx²³'s may be the same as or different from each other, and two or more Y²³'s may be the same as or different from each other]

In Formula (B-1), the descriptions for Y¹¹, Y¹², Y¹³, Y²¹, Y²², and Y²³ are the same as Y¹, Y², and Y³ in Formula (A-1) described above. Y¹¹, Y¹², and Y¹³ may be the same as or different from each other. Y²¹, Y²², and Y²³ may be the same as or different from each other. Y¹¹ and Y²¹ may be the same as or different from each other, but are preferably the same. Y¹² and Y²² may be the same as or different from each other, but are preferably the same. Y¹³ and Y²³ may be the same as or different from each other, but are preferably the same. Y¹¹, Y¹², Y¹³, Y²¹, Y²², and Y²³ are each preferably an ethylene group or a propylene group.

In Formula (B-1), the descriptions for Rx¹², Rx¹³, Rx²², and Rx²³ are the same as Rx² and Rx³ in Formula (A-1) described above. It is preferable that Rx¹² and Rx¹³ are the same, and Rx²² and Rx²³ are the same; it is more preferable that Rx¹², Rx¹³, Rx²², and Rx²³ are all the same; and it is still more preferable that Rx¹², Rx¹³, Rx²², and Rx²³ are all hydrogen atoms or all methyl groups.

In Formula (B-1), the descriptions for Lx¹ and Lx² are the same as Lx¹ in Formula (A-1) described above. Lx¹ and Lx² may be the same as or different from each other, but are preferably the same.

In Formula (B-1), the descriptions for m01 and m02 are the same as mo in Formula (A-1) described above. m01 and m02 may be the same as or different from each other, but are preferably the same. m01 and m02 are preferably 1 or 2.

Examples of the compound (B-1) include a compound represented by General Formula (B-1-1) (hereinafter, also referred to as “compound (B-1-1)”) and a compound represented by General Formula (B-1-2) (hereinafter, also referred to as “compound (B-1-2)”). The compound (B-1-1) is a compound in which two molecules of the compound (A-1-1) are bonded through a disulfide bond. The compound (B-1-2) is a compound in which two molecules of the compound (A-1-2) are bonded through a disulfide bond.

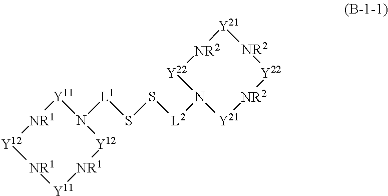

[in the formula, Y¹¹, Y¹², Y²¹, and Y²² each independently represent an alkylene group having 1 to 5 carbon atoms, R¹ and R² each independently represent a hydrogen atom or an alkyl group having 1 to 3 carbon atoms, and L¹ and L² each independently represent an alkylene group having 1 to 10 carbon atoms, which may contain at least one selected from the group consisting of an oxygen atom and a nitrogen atom]

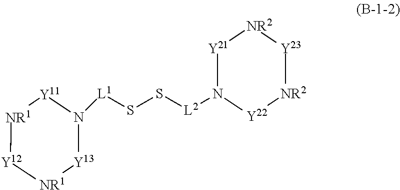

[in the formula, Y¹¹, Y¹², Y¹³, Y²¹, and Y²² each independently represent an alkylene group having 1 to 5 carbon atoms, R¹ and R² each independently represent a hydrogen atom or an alkyl group having 1 to 3 carbon atoms, and L¹ and L² each independently represent an alkylene group having 1 to 10 carbon atoms, which may contain at least one selected from the group consisting of an oxygen atom and a nitrogen atom]

In Formula (B-1-1), the descriptions for Y¹¹, Y¹², Y²¹, and Y²² are the same as Y¹ and Y² in Formula (A-1-1) described above. Two Y¹¹'s may be the same as or different from each other, but are preferably the same. Two Y¹²'s may be the same as or different from each other, but are preferably the same. Two Y²¹'s may be the same as or different from each other, but are preferably the same. Two Y²²'s may be the same as or different from each other, but are preferably the same.

Y¹¹ and Y²¹ may be the same as or different from each other, but are preferably the same. Y¹² and Y²² may be the same as or different from each other, but are preferably the same.

It is preferable that two Y¹¹'s and two Y²¹'s are all the same. It is preferable that two Y¹²'s and two Y²²'s are all the same.

Y¹¹ and Y²¹ are preferably linear alkylene groups having 2 or 3 carbon atoms, and more preferably propylene groups.

Y¹² and Y²² are preferably linear alkylene groups having 2 or 3 carbon atoms, and more preferably ethylene groups.

In Formula (B-1-2), the descriptions for Y¹¹, Y¹², Y¹³, Y²¹, Y²², and Y²³ are the same as Y¹ to Y³ in Formula (A-1-2) described above. Y¹¹, Y¹², and Y¹³ may be the same as or different from each other, but are preferably the same. Y²¹, Y²², and Y²³ may be the same as or different from each other, but are preferably the same. Y¹¹ and Y²¹ may be the same as or different from each other, but are preferably the same. Y¹² and Y²² may be the same as or different from each other, but are preferably the same. Y¹³ and Y²³ may be the same as or different from each other, but are preferably the same. It is preferable that Y¹¹, Y¹², Y¹³, Y²¹, Y²², and Y²³ are all the same. Y¹¹, Y¹², Y¹³, Y²¹, Y²², and Y²³ are preferably ethylene groups or propylene groups, and more preferably ethylene groups.

In Formulae (B-1-1) and (B-1-2), the descriptions for R¹ and R² are the same as R in Formulae (A-1-1) and (A-1-2) described above. In Formula (B-1-1), three R¹'s and three R²'s may be the same as or different from each other, but are preferably the same.

In Formula (B-1-1), it is preferable that three R¹'s are the same and three R²'s are the same; it is more preferable that three R¹'s and three R²'s are all the same; and it is still more preferable that three R¹'s and three R²'s are all hydrogen atoms or all methyl groups. The same applies to R¹ and R² in Formula (B-1-2).

In Formulae (B-1-1) and (B-1-2), the descriptions for L¹ and L² are the same as L in Formulae (A-1-1) and (A-1-2) described above. L¹ and L² in Formula (B-1-1) may be the same as or different from each other, but are preferably the same. L¹ and L² in Formula (B-1-2) may be the same as or different from each other, but are preferably the same.

Examples of the compound (B-1-1) include a compound represented by General Formula (B-1-1a) (hereinafter, also referred to as “compound (B-1-1a)”). The compound (B-1-1a) is a compound in which two molecules of the compound (A-1-1a) are bonded through a disulfide bond.

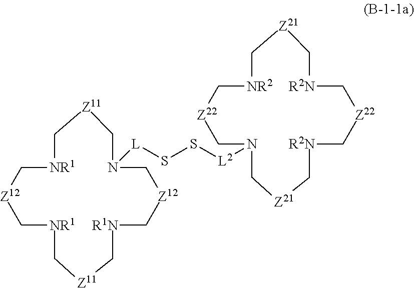

[in the formula, Z¹¹, Z¹², Z²¹, and Z²² each independently represent a single bond or a methylene group, R¹ and R² each independently represent a hydrogen atom or an alkyl group having 1 to 3 carbon atoms, and L¹ and L² each independently represent an alkylene group having 1 to 10 carbon atoms, which may contain at least one selected from the group consisting of an oxygen atom and a nitrogen atom]

The two compounds (A-1-1a) linked by a disulfide bond may be the same as or different from each other, but are preferably the same.

In General Formula (B-1-1a), the descriptions for Z¹¹ and Z²¹ are the same as Z¹ in General Formula (A-1-1a) described above. Two Z¹¹'s and two Z²¹'s may be the same as or different from each other, but are preferably the same. It is preferable that two Z¹¹'s are the same and two Z²¹'s are the same; it is more preferable that two Z¹¹'s and two Z²¹'s are all the same; and it is still more preferable that two Z¹¹'s and two Z²¹'s are all methylene groups.

In General Formula (B-1-1a), the descriptions for Z¹² and Z²² are the same as Z² in General Formula (A-1-1a) described above. Two Z¹²'s and two Z²²'s may be the same as or different from each other, but are preferably the same. It is preferable that two Z¹²'s are the same and two Z²²'s are the same; it is more preferable that two Z¹²'s and two Z²²'s are all the same; and it is still more preferable that two Z¹²'s and two Z²²'s are all single bonds.

In General Formula (B-1-1a), the descriptions for R¹ and R² are the same as R in General Formula (A-1-1a) described above. Three R¹'s and three R²'s may be the same as or different from each other, but are preferably the same. It is preferable that three R¹'s are the same and three R²'s are the same; it is more preferable that three R¹'s and three R²'s are all the same; and it is still more preferable that three R¹'s and three R²'s are all hydrogen atoms or all methyl groups.

In General Formula (B-1-1a), the descriptions for L¹ and L² are the same as L in General Formula (A-1-1a) described above. L¹ and L² may be the same as or different from each other, but are preferably the same.

As the compound (B-1-1a), a compound represented by General Formula (B-1-1b) (hereinafter, also referred to as “compound (B-1-1b)”) is preferable. The compound (B-1-1b) is a compound in which two molecules of the compound (A-1-1b) are bonded through a disulfide bond.

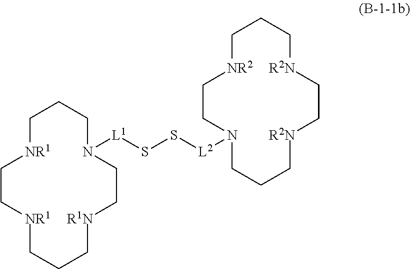

[in the formula, R¹ and R² each independently represent a hydrogen atom or an alkyl group having 1 to 3 carbon atoms, and L¹ and L² each independently represent an alkylene group having 1 to 10 carbon atoms, which may contain at least one selected from the group consisting of an oxygen atom and a nitrogen atom]

R¹ and R² in Formula (B-1-1b) are the same as R¹ and R² in Formula (B-1-1a) described above. R¹ and R² are preferably a hydrogen atom or a methyl group, and more preferably a hydrogen atom.

L¹ and L² in Formula (B-1-1b) are the same as L¹ and L² in Formula (B-1-1a) described above. L¹ and L² are each preferably an alkylene group having 1 to 6 carbon atoms or a polyoxyalkylene group having 1 to 6 carbon atoms; more preferably an alkylene group having 1 to 6 carbon atoms, a polyoxyethylene group having 1 to 6 carbon atoms, or a polyoxypropylene group having 1 to 6 carbon atoms; still more preferably an ethylene group or a propylene group; and particularly preferably an ethylene group.

Specific examples of the compound (B-1-1b) include a compound represented by Formula (B-1-1-1) (hereinafter, also referred to as “compound (B-1-1-1)”). The compound (B-1-1-1) is a compound in which two molecules of the compound (A-1-1-1) are bonded through a disulfide bond.

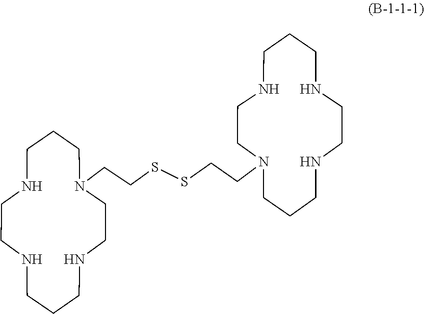 [SMILES: C1CNCCNCCCN(CCSSCCN2CCCNCCNCCCNCC2)CCNC1]

Specific examples of the compound (B-1-2) include a compound represented by Formula (B-1-2-1) (hereinafter, also referred to as “compound (B-1-2-1)”). The compound (B-1-2-1) is a compound in which two molecules of the compound (A-1-2-1) are bonded through a disulfide bond.

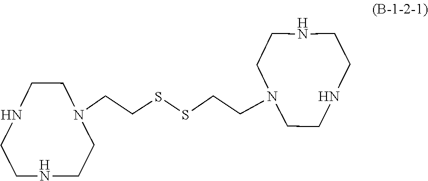 [SMILES: C1CNCCN(CCSSCCN2CCNCCNCC2)CCN1]

### <Compound (C)>

The compound (C) is a complex of the compound (A) or the compound (B) with a transition metal ion. Examples of the transition metal ion include ions of elements located between Group 3 and Group 12 in the periodic table. Examples of the transition metal ion include, but are not limited to, copper ions (for example, Cu(II) ions and Cu(I) ions), nickel ions (for example, Ni(II) ions), iron ions (for example, Fe(II) ions and Fe(III) ions), manganese ions (for example, Mn(II) ions and Mn(III) ions), zinc ions (for example, Zn(II) ions), and molybdenum ions (for example, Mo (IV) ions and Mo (VI) ions).

Examples of the complex of the compound (A) with a transition metal ion include a complex formed by coordination of the transition metal ion to a polyamine ring of the compound (A), and a complex formed by coordination of the transition metal ion to two polyamine rings of the compound (B).

### <Compound (D)>

The compound (D) is a salt of the compound (A) or the compound (B). The above-described salt is not particularly limited, and may be a salt with an acid or a salt with a base. Examples of the salt with an acid include salts with an inorganic acid such as hydrochloric acid, phosphoric acid, nitric acid, sulfuric acid, and sulfurous acid; and salts with an organic acid such as formic acid, acetic acid, propionic acid, butyric acid, oxalic acid, malonic acid, succinic acid, maleic acid, fumaric acid, tartaric acid, malic acid, mandelic acid, methanesulfonic acid, p-toluenesulfonic acid, and trifluoroacetic acid. Examples of the salt with a base include salts with an alkali metal such as sodium and potassium; salts with an alkaline earth metal such as magnesium and calcium; and ammonium salts.

### <Compound (E)>

The compound (E) is a solvate of the compound (A) or the compound (B). Examples of the solvate include hydrates, and solvates with an organic solvent such as alcohol (for example, methanol, ethanol, propanol, and isopropanol), acetone, tetrahydrofuran, dioxane, DMF, and DMSO.

The term ‘protein folding agent’ means an agent that promotes protein folding. The “promoting protein folding” means that, compared with the absence of the protein folding agent, a greater amount of native structural protein (folded protein) is produced when a folding reaction of a reduced and denatured protein is performed in the presence of the protein folding agent. The protein folding agent is, for example, an agent that, when a reduced and denatured protein (for example, 30 μM) and the protein folding agent are dissolved in a Tris-HCl buffer (50 mM Tris-HCl, 300 mM NaCl, pH: 7.5) and incubated at 30° C. for 1 hour, provides a formation proportion of the native structural protein of 5% or more, preferably 10% or more. The protein folding agent according to the present embodiment can be used for folding a protein as a native structural protein having one or more disulfide bonds.

The protein folding agent according to the present embodiment preferably includes at least one compound selected from the group consisting of the compound (B), a metal complex of the compound (B), a salt of the compound (B), and a solvate of the compound (B) (hereinafter, referred to as “compound (B) and the like”); and preferably includes the compound (B).

In addition to the compound (B) and the like, the protein folding agent according to the present embodiment preferably includes at least one compound selected from the group consisting of the compound (A), a metal complex of the compound (A), a salt of the compound (A), and a solvate of the compound (A) (hereinafter, referred to as “compound (A) and the like”); and preferably includes the compound (A) and the compound (B).

When the protein folding agent according to the present embodiment includes the compound (A) and the like, and the compound (B) and the like, a ratio of the compound (A) and the like and the compound (B) and the like is not particularly limited. A molar ratio of the compound (A) and the like and the compound (B) and the like includes, for example, 1:10 to 20:1, preferably 1:5 to 15:1, more preferably 1:3 to 10:1, still more preferably 1:2 to 10:1, even more preferably 1:1 to 8:1, and particularly preferably 2:1 to 7:1. When the compound (A) is the compound (A-1-2) and the compound (B) is the compound (B-1-2), the molar ratio of the compound (A) and the like and the compound (B) and the like includes, for example, 1:10 to 30:1, preferably 1:5 to 20:1, more preferably 1:3 to 20:1, still more preferably 1:2 to 15:1, even more preferably 1:1 to 15:1, and particularly preferably 2:1 to 10:1.

The protein folding agent according to the present embodiment may include at least one compound selected from the group consisting of the compound (A-2), a metal complex of the compound (A-2), a salt of the compound (A-2), and a solvate of the compound (A-2) (hereinafter, referred to as “compound (A-2) and the like”); and preferably includes the compound (A-2).

### (Optional Component)

The protein folding agent according to the present embodiment may include an optional component in addition to the compound (A) and the like and/or the compound (B) and the like.

Examples of the optional component include oxidizing agents and/or reducing agents, other than the compound (A) and the like and the compound (B) and the like.

Examples of the reducing agent include compounds having a thiol group. Examples of the reducing agent include reduced glutathione (GSH), 1,4-dithiothreitol, (+)-trans-1,2-bis(2-mercaptoacetamido)cyclohexane (BMC) (K. J. Woycechowsky et al., Chem. Biol., 1999, 6, 871-879), aromatic thiols (D. J. Madar et al., J. Biotech., 2009, 142, 214-219), cyclic selenoxide (K. Arai, K et al., Chem. Eur. J., 2011, 17, 481-485), peptides having a Cys-XX-Cys structure (X is any amino acid; Cys is cysteine) (W. J. Lees et al., Curr. Opin. Chem. Biol., 2008, 12, 740-745), selenoglutathione (J. Beld et al., Biochemistry, 2007 May 8; 46 (18): 5382-90), selenol-containing peptides (S. Tsukagoshi et al., Chem. Asian J., 2020 Sep. 1; 15 (17): 2646-52), and thiol compounds described in JP-A-2022-135954. Examples of the oxidizing agent include compounds having a disulfide bond. Examples of the oxidizing agent include oxidized compounds of the above-described reducing agents.

The protein folding agent preferably includes the reducing agent, and more preferably includes both the reducing agent and the oxidizing agent.

When the protein folding agent includes at least one compound selected from the group consisting of the compound (A-1), a metal complex of the compound (A-1), a salt of the compound (A-1), and a solvate of the compound (A-1) (hereinafter, referred to as “compound (A-1) and the like”), it is preferable that the protein folding agent includes, as an oxidizing agent, the compound (A-2) and the like, the compound (B) and the like, or an oxidizing agent other than the compound (B) and the like.

When the protein folding agent includes the compound (A-2) and the like or the compound (B) and the like, the protein folding agent may include, as a reducing agent, the compound (A-1) and the like or a reducing agent other than the compound (A-1) and the like.

The protein folding agent according to the present embodiment can suppress aggregation of proteins and promote protein folding in the presence of a transition metal ion. The protein folding agent according to the present embodiment can further promote protein folding in the presence of a transition metal, compared to in the absence of a transition metal ion. Therefore, the protein folding agent according to the present embodiment can be suitably used for protein folding in the presence of a transition metal ion. Examples of the above-described transition metal include ions of elements located between Group 3 and Group 12 in the periodic table. Examples of the transition metal ion include, but are not limited to, copper ions (for example, Cu(II) ions and Cu(I) ions), nickel ions (for example, Ni(II) ions), iron ions (for example, Fe(II) ions and Fe(III) ions), manganese ions (for example, Mn(II) ions and Mn(III) ions), zinc ions (for example, Zn(II) ions), and molybdenum ions (for example, Mo (IV) ions and Mo (VI) ions). The above-described transition metal ion is preferably at least one selected from the group consisting of copper ions (for example, Cu(II) ions and Cu(I) ions), nickel ions (for example, Ni(II) ions), and zinc ions (for example, Zn(II) ions).

There are proteins that contain metal ions within the molecule (metalloproteins). For example, among enzymes that contain metal ions, some contain metal ions in their active centers. When chemically or biologically synthesizing such metalloproteins and folding the proteins into a native structure, it is necessary to perform a folding reaction in the presence of the metal ions. However, in the presence of transition metal ions, there is a risk of protein aggregation, so reaction conditions need to be strictly controlled.

The protein folding agent according to the present embodiment can suppress protein aggregation and promote folding into a native structural protein in the presence of a transition metal ion. Therefore, the protein folding agent according to the present embodiment can be suitably used for folding a protein containing a transition metal ion.

Examples of the transition metal ion contained in the protein include, but are not limited to, a copper ion, a nickel ion, an iron ion, a manganese ion, a zinc ion, and a molybdenum ion. Examples of the protein containing a copper ion include Cytochrome c oxidase. Examples of the protein containing a nickel ion include Urease. Examples of the protein containing an iron ion include Catalase, Cytochrome (heme), Nitrogenase, and Hydrogenase. Examples of the protein containing a manganese ion include Arginase. Examples of the protein containing a zinc ion include alcohol dehydrogenase, carbonic anhydrase, and DNA polymerase. Examples of the protein containing molybdenum include nitrate reductase.

The protein folding agent according to the present embodiment can be used for folding of an unfolded protein or refolding of a misfolded protein.

The unfolded protein refers to a protein that is not folded. In the unfolded protein, disulfide bonds formed in a native structural protein are reduced to thiol groups. A protein reduced and denatured by a reducing agent is the unfolded protein.

The misfolded protein refers to a protein in which one or more disulfide bonds are formed in a structure other than a thermodynamically most stable native structure.

The unfolded protein and the misfolded protein cannot express activity possessed by the native structural protein. By allowing the protein folding agent according to the present embodiment to act on such unfolded protein or misfolded protein, it is possible to fold the protein into a thermodynamically most stable native structural protein.

### [Compound]

A second aspect of the present disclosure is the above-described compound (B-1), the above-described compound (A-1-2), or the above-described compound (A-2). The description of the compound (B-1), the compound (A-1-2), or the compound (A-2-1) is as described above. These compounds can be used as the protein folding agent according to the first aspect as described above.

### <Method for Producing Compound (B-1)>

The compound (B-1) can be produced by combining known methods. For example, the compound (B-1) in which Rx¹², Rx¹³, Rx²², and Rx²³ are hydrogen atoms can be synthesized by the following synthesis scheme. In the following synthesis scheme, TsCl represents p-toluenesulfonyl chloride, Boc₂O represents di-tert-butyl dicarbonate, Ts represents a p-toluenesulfonyl group, and Boc represents a tert-butoxycarbonyl group.

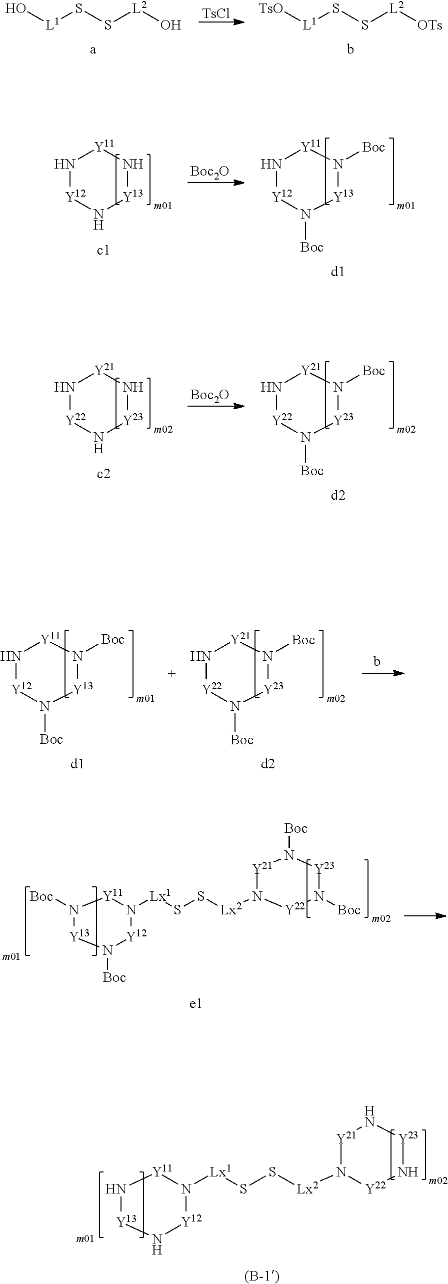

[in the formulae, Y¹¹, Y¹², Y¹³, Y²¹, Y²², and Y²³ each independently represent an alkylene group having 1 to 5 carbon atoms, Lx¹ and Lx² each independently represent an alkylene group having 1 to 10 carbon atoms, which may contain at least one selected from the group consisting of an oxygen atom and a nitrogen atom, m01 and m02 each independently represent an integer of 1 to 3, when m01 is an integer of 2 or more, two or more Y¹³'s may be the same as or different from each other, and when m02 is an integer of 2 or more, two or more Y²³'s may be the same as or different from each other]

The reaction of obtaining the compound b by the reaction of the compound a and TsCl can be performed in the presence of a base such as triethylamine. As a reaction solvent, for example, dichloromethane or the like can be used. Examples of the reaction temperature include 0° C. to 30° C. Examples of the reaction time include 20 to hours. For example, the method described in P. Jain et al. (Langmuir 2019, 35, 1864) can be referred to.

The reaction of obtaining the compound d1 by the reaction of the compound c1 and Boc₂O, and the reaction of obtaining the compound d2 by the reaction of the compound c2 and Boc₂O can be performed in the presence of a base such as triethylamine. As a reaction solvent, for example, dichloromethane or the like can be used. Examples of the reaction temperature include −15° C. to 30° C. Examples of the reaction time include 20 to 30 hours. For example, the method described in Boitrel et al. (Tetrahedron Lett. 1995, 36, 4995.) can be referred to.

The reaction of obtaining the compound e1 by the reaction of the compound d1, the compound d2, and the compound b can be performed in the presence of a base such as potassium carbonate. As a reaction solvent, for example, dehydrated acetonitrile or the like can be used. Examples of the reaction temperature include 40° C. to 80° C. Examples of the reaction time include 10 to 40 hours. The reaction can be performed under a nitrogen atmosphere.

The reaction of obtaining the compound (B-1) (compound represented by General Formula (B-1′)) by deprotection of Boc of the compound e1 can be performed under acidic conditions using concentrated hydrochloric acid or the like. As a reaction solvent, an ethanol solution can be used. Examples of the reaction temperature include 10° C. to 40° C. Examples of the reaction time include 1 to 10 hours. The reaction can be performed under an air atmosphere.

For example, the compound (B-1) in which Rx¹², Rx¹³, Rx²², and Rx²³ are methyl groups and Lx¹ and Lx² are alkylene groups having 2 to 10 carbon atoms (which may contain at least one selected from the group consisting of an oxygen atom and a nitrogen atom) can be synthesized by the following synthesis scheme (J. A. Halfen et al., Chem. Commun. 2003, 2894-2895). In the following synthesis scheme, TsCl represents p-toluenesulfonyl chloride, EtOTFA represents ethyl trifluoroacetate, Ts represents a p-toluenesulfonyl group, and TFA represents a trifluoroacetyl group.

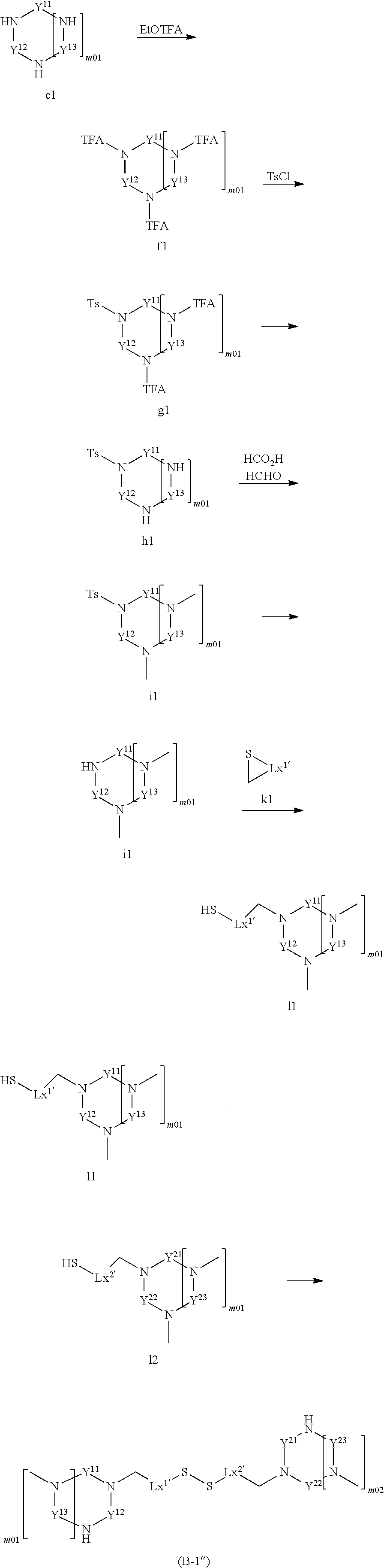

[in the formulae, Y¹¹, Y¹², Y¹³, Y²¹, Y²², and Y²³ each independently represent an alkylene group having 1 to 5 carbon atoms, Lx¹′ and Lx²′ represent an alkylene group having 1 to 9 carbon atoms, which may contain at least one selected from the group consisting of an oxygen atom and a nitrogen atom, m01 and m02 each independently represent an integer of 1 to 3, when m01 is an integer of 2 or more, two or more Y¹³'s may be the same as or different from each other, and when m02 is an integer of 2 or more, two or more Y²³'s may be the same as or different from each other]

The reaction of obtaining the compound f1 by the reaction of the compound c1 and EtOTFA can be performed in the presence of a base such as triethylamine. As a reaction solvent, for example, methanol or the like can be used. Examples of the reaction temperature include 0° C. to 40° C. Examples of the reaction time include 10 to hours.

The reaction of obtaining the compound g1 by the reaction of the compound f1 and TsCl can be performed in the presence of a base such as triethylamine. As a reaction solvent, for example, dichloromethane or the like can be used. The reaction can be performed by refluxing the reaction solution for 10 to 30 hours.

The reaction of obtaining the compound h1 by hydrolysis of the compound g1 can be performed in the presence of a base such as potassium hydroxide. As a reaction solvent, for example, methanol or the like can be used. The reaction can be performed by refluxing the reaction solution for 1 to 20 hours.

The reaction of obtaining the compound i1 by methylation of the compound h1 can be performed using formic acid and formaldehyde. The reaction can be performed by refluxing the reaction solution for 1 to 20 hours.

The reaction of obtaining the compound j1 by detosylation of the compound i1 can be performed in the presence of a strong acid such as hydrobromic acid. As a reaction solvent, for example, phenol can be used. Examples of the reaction temperature include 60° C. to 120° C. Examples of the reaction time include 10 to 40 hours.

The reaction of obtaining the compound l1 by the reaction of the compound j1 and the compound k1 can be performed using a reaction solvent such as benzene. Examples of the reaction temperature include 50° C. to 150° C. Examples of the reaction time include 10 to 40 hours.

The compound 12 can be obtained by the same method as described above.

The reaction of obtaining the compound (B-1) (compound represented by General Formula (B-1″)) by oxidation reaction of the compound 11 and the compound 12 can be performed using an oxidizing agent such as oxidized DTT. As a reaction solvent, water can be used. Examples of the reaction temperature include 0° C. to 40° C. Examples of the reaction time include 1 to 30 hours.

After completion of each reaction, isolation and purification operations of each compound in the reaction solution may be performed.

For the isolation and purification, conventionally known methods can be used; and for example, concentration, solvent extraction, distillation, crystallization, recrystallization, chromatography, and the like can be used alone or a combination of two or more thereof can be used.

The structure of the compound obtained by each reaction can be confirmed by general organic analysis methods such as ¹H-nuclear magnetic resonance (NMR) spectroscopy and MALDI-TOF mass spectrometric spectrum measurement.

### (Method for Producing Compound (B-1-1a))

An example of a method for producing the compound (B-1-1a) is shown below as the example of the compound (B-1). The compound (B-1-1a) can be produced by combining known methods. For example, the compound (B-1-1a) in which R¹ and R² are hydrogen atoms can be synthesized by the following synthesis scheme. In the following synthesis scheme, TsCl represents p-toluenesulfonyl chloride, Boc₂O represents di-tert-butyl dicarbonate, Ts represents a p-toluenesulfonyl group, and Boc represents a tert-butoxycarbonyl group.

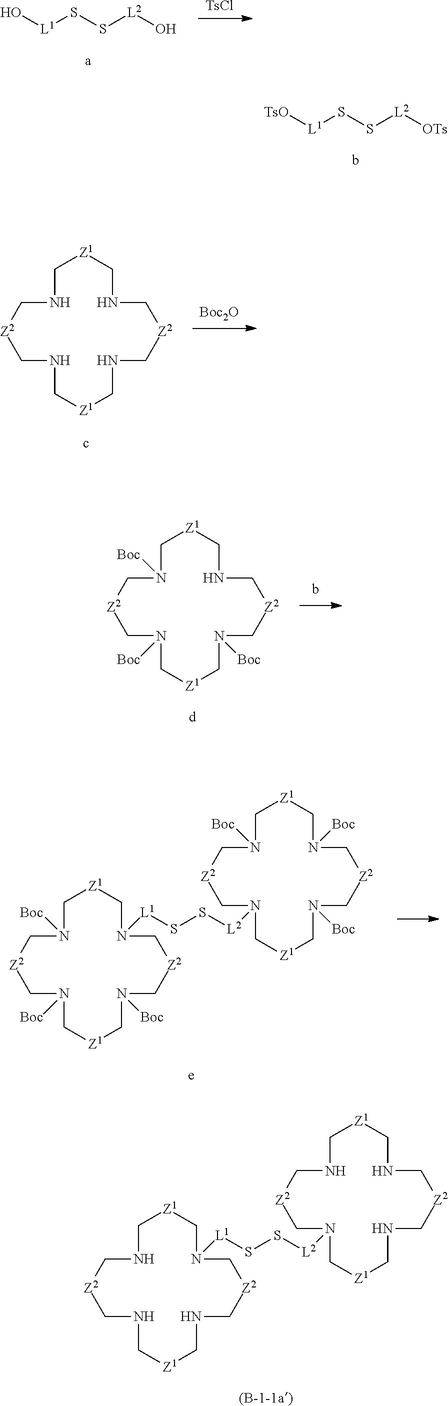

[in the formulae, Z¹ and Z² each independently represent a single bond or a methylene group, and L¹ and L² each independently represent an alkylene group having 1 to 10 carbon atoms, which may contain at least one selected from the group consisting of an oxygen atom and a nitrogen atom]

The reaction of obtaining the compound b by the reaction of the compound a and TsCl can be performed in the presence of a base such as triethylamine. As a reaction solvent, for example, dichloromethane or the like can be used. Examples of the reaction temperature include 0° C. to 30° C. Examples of the reaction time include 20 to hours. For example, the method described in P. Jain et al. (Langmuir 2019, 35, 1864) can be referred to.

The reaction of obtaining the compound d by the reaction of the compound c and Boc₂O can be performed in the presence of a base such as triethylamine. As a reaction solvent, for example, dichloromethane or the like can be used. Examples of the reaction temperature include −15° C. to 30° C. Examples of the reaction time include 20 to 30 hours. For example, the method described in Boitrel et al. (Tetrahedron Lett. 1995, 36, 4995.) can be referred to.

The reaction of obtaining the compound e by the reaction of the compound d and the compound (b) can be performed in the presence of a base such as potassium carbonate. As a reaction solvent, for example, dehydrated acetonitrile or the like can be used. Examples of the reaction temperature include 40° C. to 80° C. Examples of the reaction time include 10 to 40 hours. The reaction can be performed in a nitrogen atmosphere.

The reaction of obtaining the compound (B) (compound represented by General Formula (B-1-1a′)) by deprotection of Boc of the compound e can be performed under acidic conditions using concentrated hydrochloric acid or the like. As a reaction solvent, an ethanol solution can be used. Examples of the reaction temperature include 10° C. to 40° C. Examples of the reaction time include 1 to 10 hours. The reaction can be performed under an air atmosphere.

For example, the compound (B-1-1a) in which R¹ and R² are methyl groups and L¹ and L² are alkylene groups having 2 to 10 carbon atoms (which may contain at least one selected from the group consisting of an oxygen atom and a nitrogen atom) can be synthesized by the following synthesis scheme (J. A. Halfen et al., Chem. Commun. 2003, 2894-2895). In the following synthesis scheme, TsCl represents p-toluenesulfonyl chloride, EtOTFA represents ethyl trifluoroacetate, Ts represents a p-toluenesulfonyl group, and TFA represents a trifluoroacetyl group.

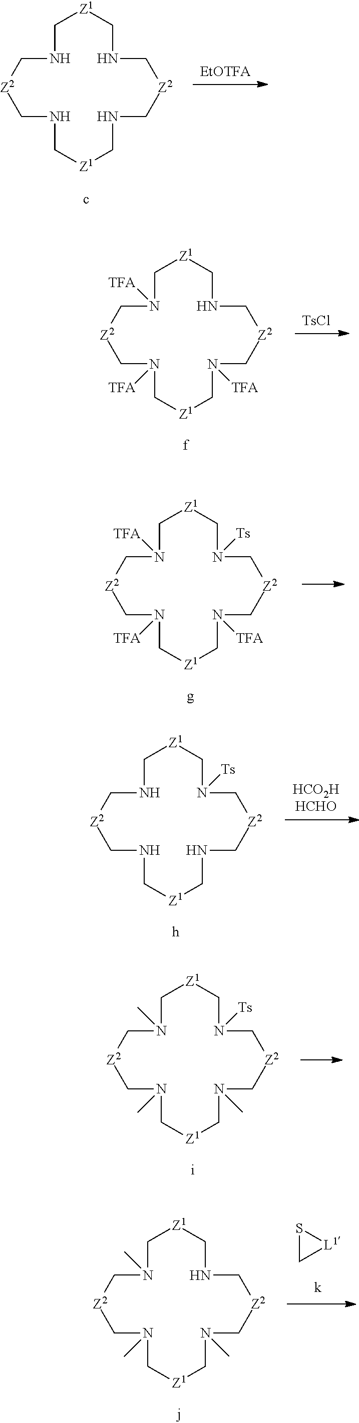

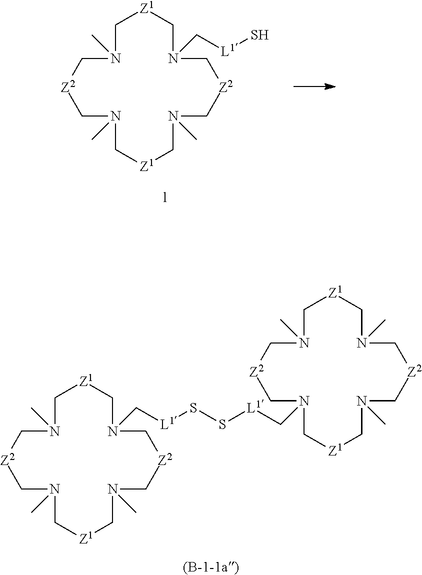

[in the formulae, Z¹ and Z² each independently represent a single bond or a methylene group, and L¹′ represents an alkylene group having 1 to 9 carbon atoms, which may contain at least one selected from the group consisting of an oxygen atom and a nitrogen atom]

The reaction of obtaining the compound f by the reaction of the compound c and EtOTFA can be performed in the presence of a base such as triethylamine. As a reaction solvent, for example, methanol or the like can be used. Examples of the reaction temperature include 0° C. to 40° C. Examples of the reaction time include 10 to hours.

The reaction of obtaining the compound g by the reaction of the compound f and TsCl can be performed in the presence of a base such as triethylamine. As a reaction solvent, for example, dichloromethane or the like can be used. The reaction can be performed by refluxing the reaction solution for 10 to 30 hours.

The reaction of obtaining the compound h by hydrolysis of the compound g can be performed in the presence of a base such as potassium hydroxide. As a reaction solvent, for example, methanol or the like can be used. The reaction can be performed by refluxing the reaction solution for 1 to 20 hours.

The reaction of obtaining the compound i by methylation of the compound h can be performed using formic acid and formaldehyde. The reaction can be performed by refluxing the reaction solution for 1 to 20 hours.

The reaction of obtaining the compound j by detosylation of the compound i can be performed in the presence of a strong acid such as hydrobromic acid. As a reaction solvent, for example, phenol can be used. Examples of the reaction temperature include 60° C. to 120° C. Examples of the reaction time include 10 to 40 hours.

The reaction of obtaining the compound 1 by the reaction of the compound j and the compound k can be performed using a reaction solvent such as benzene. Examples of the reaction temperature include 50° C. to 150° C. Examples of the reaction time include 10 to 40 hours.

The reaction of obtaining the compound (B) (compound represented by General Formula (B-1-1a″)) by oxidation reaction of the compound I can be performed using an oxidizing agent such as oxidized DTT. As a reaction solvent, water can be used. Examples of the reaction temperature include 0° C. to 40° C. Examples of the reaction time include 1 to 30 hours.

After completion of each reaction, isolation and purification operations of each compound in the reaction solution may be performed.

For the isolation and purification, conventionally known methods can be used; and for example, concentration, solvent extraction, distillation, crystallization, recrystallization, chromatography, and the like can be used alone or a combination of two or more thereof can be used.

A structure of the compound obtained by each reaction can be confirmed by general organic analysis methods such as 1H-nuclear magnetic resonance (NMR) spectroscopy and MALDI-TOF mass spectrometric spectrum measurement.

The compound (B-1-1) and the compound (B-1-2) can also be produced in the same manner.

<Method for producing compound (A-1-2)>

The compound (A-1-2) can be obtained by performing a reduction reaction of the compound (B-1-2). The reduction reaction of the compound (B-1-2) can be performed, for example, under acidic conditions using concentrated hydrochloric acid or the like. As a reaction solvent, an ethanol solution can be used. Examples of the reaction temperature include 10° C. to 40° C. Examples of the reaction time include 1 to hours. The reaction can be performed under an air atmosphere. As the reducing agent, for example, dithiothreitol (DTT) or the like can be used.

### <Method for Producing Compound (A-2)>

The compound (A-2) can be produced by combining known methods. The compound (A-2) can be synthesized, for example, by the following synthesis scheme. In the following synthesis scheme, NaOH aq represents a sodium hydroxide aqueous solution.

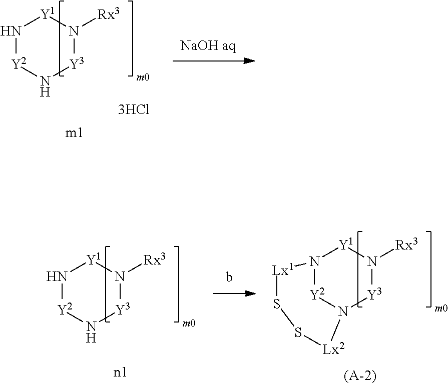

[in the formulae, Rx³ represents a hydrogen atom or a monovalent organic group, Y¹ to Y³ each independently represent an alkylene group having 1 to 5 carbon atoms, Lx¹ and Lx² each independently represent an alkylene group having 1 to 10 carbon atoms, which may contain at least one selected from the group consisting of an oxygen atom and a nitrogen atom, m0 represents an integer of 1 to 3, and when m0 is an integer of 2 or more, two or more Rx³'s may be the same as or different from each other, and two or more Y³'s may be the same as or different from each other].

The reaction of obtaining the compound n1 from the compound ml can be performed in the presence of a base such as sodium hydroxide. Examples of the reaction temperature include 0° C. to 30° C. Examples of the reaction time include 20 to hours. For example, the methods described in P. Jain et al. (Langmuir 2019, 35, 1864) and C. A. Barta et al (Inorg Chem. 2008, 47, 2280.) can be referred to.

The reaction of obtaining the compound (A-2) by the reaction of the compound nl and the compound b can be performed in the presence of a base such as potassium carbonate. As a reaction solvent, for example, dehydrated acetonitrile or the like can be used. Examples of the reaction temperature include 40° C. to 80° C. Examples of the reaction time include 10 to 40 hours. The reaction can be performed in a nitrogen atmosphere.

After completion of each reaction, isolation and purification operations of each compound in the reaction solution may be performed.

For the isolation and purification, conventionally known methods can be used; and for example, concentration, solvent extraction, distillation, crystallization, recrystallization, chromatography, and the like can be used alone or a combination of two or more thereof can be used.

A structure of the compound obtained by each reaction can be confirmed by general organic analysis methods such as 1H-nuclear magnetic resonance (NMR) spectroscopy and MALDI-TOF mass spectrometric spectrum measurement.

### [Protein Folding Method]

A third aspect of the present disclosure is a protein folding method. The protein folding method according to the present aspect includes a step of incubating a protein in the presence of the protein folding agent according to the first aspect.

### <Step of Incubating Protein; Protein Folding Step>

The incubation of the protein can be performed by dissolving the protein and the protein folding agent according to the first aspect (for example, the compound (A) and the like, and the compound (B) and the like) in a suitable buffer to prepare a folding reaction solution, and then incubating the solution. By performing the incubation, a protein folding reaction can be carried out.

The buffer is not particularly limited as long as the protein folding reaction proceeds, and buffers usually used in the biochemical field can be used without particular limitation. Examples of the buffer include amine-based buffers such as Tris-HCl buffer, Tris buffer, MES buffer, and Tricine buffer; phosphate buffers; and Good's buffers. Examples of a pH of the buffer include 4 to 10. The pH of the buffer is preferably 5 to 9, more preferably 7 to 9, and still more preferably 7 to 8. As the buffer, a Tris-HCl buffer to which sodium chloride is added is preferable. Specific examples of the buffer include a buffer containing 50 mM tris (hydroxymethyl)aminomethane and 30 mM sodium chloride and adjusted to a pH of 7.5 with hydrochloric acid.

The concentration of the protein folding agent in the folding reaction solution is not particularly limited. When the protein folding agent includes the compound (A) and the like, the concentration of the compound (A) and the like includes, for example, 1 μM to 1000 mM, preferably 10 μM to 100 mM, more preferably 300 μM to 50 mM, still more preferably 500 μM to 10 mM, and particularly preferably 800 μM to 5 mM. When the protein folding agent includes the compound (B) and the like, the concentration of the compound (B) and the like includes, for example, 1 μM to 1000 mM, preferably 10 μM to 100 mM, more preferably 50 μM to 1 mM, still more preferably 100 to 500 μM, and particularly preferably 100 to 300 μM. When the protein folding agent includes the compound (A) and the like and the compound (B) and the like, a molar ratio of the compound (A) and the like and the compound (B) and the like includes, for example, 1:10 to 20:1, preferably 1:5 to 15:1, more preferably 1:3 to 10:1, still more preferably 1:2 to 10:1, even more preferably 1:1 to 8:1, and particularly preferably 2:1 to 7:1. When the compound (A) is the compound (A-1-2) and the compound (B) is the compound (B-1-2), the molar ratio of the compound (A) and the like and the compound (B) and the like includes, for example, 1:10 to 30:1, preferably 1:5 to 20:1, more preferably 1:3 to 20:1, still more preferably 1:2 to 15:1, even more preferably 1:1 to 15:1, and particularly preferably 2:1 to 10:1.

Examples of the protein used for the incubation include a protein having one or more disulfide bonds. The number of disulfide bonds in the protein is not particularly limited as long as it is 1 or more. Examples of the number of disulfide bonds in the protein include 1 to 20, 1 to 15, 1 to 10, or 1 to 5.

The molecular weight of the protein is not particularly limited, but for example, includes 1,000 to 10,000,000, and preferably 1,000 to 250,000.

As the protein, an unfolded protein or a misfolded protein may be used. For example, a protein reduced and denatured with a reducing agent may be used.

The protein folding agent according to the first aspect promotes protein folding particularly in the presence of a transition metal ion. Therefore, the protein is preferably a protein containing a transition metal ion in a native structural form.

Specific examples of the transition metal ion and the protein containing a transition metal ion include the same as those described above.

A protein concentration in the folding reaction solution is not particularly limited and can be appropriately set according to the type of protein. Examples of the protein concentration, for example, the concentration in the folding reaction solution, include 0.1 to 1000 μM, preferably 0.5 to 500 μM, more preferably 1 to 300 μM, and still more preferably 5 to 100 μM.

The folding reaction is preferably performed in the presence of a transition metal ion. By performing the folding reaction in the presence of a transition metal ion, the protein containing a transition metal can be obtained. The folding reaction solution preferably contains a transition metal ion. The transition metal ion can be added to the folding reaction solution, for example, in the state of a transition metal salt. Examples of the transition metal salt include, but are not limited to, hydrochlorides, sulfates, nitrates, carbonates, and phosphates. Specific examples of a salt of the copper ion include copper (II) chloride (CuCl₂) and copper (II) sulfate (CuSO₄). Specific examples of a salt of the nickel ion include nickel (II) chloride (NiCl₂) and nickel (II) sulfate (NiSO₄). Specific examples of a salt of the iron ion include iron (II) chloride (FeCl₂) and iron (II) sulfate (FeSO₄). Specific examples of a salt of the manganese ion include manganese (II) chloride (MnCl₂) and manganese (II) sulfate (MnSO₄). Specific examples of a salt of the zinc ion include zinc (II) chloride (ZnCl₂) and zinc (II) sulfate (ZnSO₄). Specific examples of a salt of the molybdenum ion include molybdenum (V) chloride (Mo₂Cl₁₀).

The concentration of the transition metal ion in the folding reaction solution is not particularly limited, but examples thereof include 10 to 1000 μM, preferably 10 to 500 μM, and more preferably 20 to 300 μM. When the protein contains the transition metal ion in the molecule, the molar concentration of the transition metal ion is preferably higher than the molar concentration of the protein, and may be, for example, 1.2 to 20 times the molar concentration of the protein.

When the folding reaction is performed in the presence of a transition metal ion, the concentration of the compound (A) and the like and the compound (B) and the like in the folding reaction solution may be a catalytic amount. For example, the concentration of the compound (A) and the like and the compound (B) and the like includes 0.01 to 20 μM, preferably 0.1 μM to 10 μM, more preferably 0.5 μM to 10 μM, and still more preferably 1 to 10 μM.

The incubation temperature is not particularly limited as long as the protein folding reaction can proceed. The incubation temperature can be appropriately selected according to the type of protein. Examples of the incubation temperature include 10° C. to 50° C., preferably 20° C. to 40° C., and more preferably 25° C. to 35° C.

The incubation time can be appropriately selected according to the type of protein. Examples of the incubation time include 1 to 300 minutes, preferably 10 to 200 minutes, and more preferably 30 to 100 minutes. The folding state may be observed over time, and the incubation may be terminated at the time point when the most stable native structure is attained. For observation of the folding state, for example, a thiol group modifier such as 4-acetamido-4′-maleimidylstilbene-2,2′-disulfonic acid (AMS) can be used.

### <Optional Step>

The method according to the present aspect may include an optional step in addition to the above-described incubation step. Examples of the optional step include a step of unfolding the protein, a step of isolating the folded protein, and the like.

### (Protein Unfolding Step)

The method according to the present aspect may include, before the above-described incubation step (protein folding step), a step of unfolding the protein.

The step of unfolding the protein can be performed by incubating the protein in the presence of a reducing agent. Examples of the reducing agent include reduced glutathione (GSH), β-mercaptoethanol, and 1,4-dithiothreitol. For unfolding of the protein, a protein denaturant (for example, guanidine hydrochloride, urea, and the like) may be used.

The protein unfolding reaction may be performed by dissolving the protein, the reducing agent, and the protein denaturant in a medium such as water and a buffer to prepare a protein unfolding reaction solution, and then incubating the solution.

Examples of the protein concentration in the unfolding reaction solution include 0.1 to 100 mg/mL.

Examples of the concentration of the reducing agent in the unfolding reaction solution include 0.1 to 1000 mM. The concentration of the oxidizing agent is preferably 1 to 500 mM, more preferably 10 to 300 mM, and still more preferably 50 to 200 mM.

Examples of the concentration of the denaturant in the unfolding reaction solution include 0.1 to 100 M. The concentration of the denaturant is preferably 0.5 to 50 M, more preferably 1 to 30 M, and still more preferably 1 to 20 M.

Examples of the pH of the unfolding reaction solution include 5 to 10, preferably 6 to 9.5, more preferably 7 to 9, and still more preferably 8 to 9.

Examples of a buffer usable for the unfolding reaction solution include the same as those described above. Specific examples of the buffer include 0.1 M Tris-HCl buffer.

The incubation temperature is not particularly limited as long as the protein unfolding reaction can proceed. The incubation temperature can be appropriately selected according to the type of protein. Examples of the incubation temperature include 10° C. to 60° C., preferably 20° C. to 55° C., and more preferably 20° C. to 50° C.

The incubation time can be appropriately selected according to the type of protein. Examples of the incubation time include 1 to 600 minutes, preferably 10 to 500 minutes, more preferably 30 to 400 minutes, and still more preferably 60 to 300 minutes. The unfolding state may be observed over time, and the incubation may be terminated at the time point when all disulfide bonds are reduced.

After the incubation, the denaturant, the reducing agent, and the like may be removed by, for example, dialyzing the unfolding reaction solution.

### (Protein Isolation Step)

The method according to the present aspect may include, after the above-described incubation step (protein folding step), a step of isolating the folded protein. For isolation of the protein, known protein isolation methods can be used without particular limitation. Examples of the protein isolation method include salting out, dialysis, and column chromatography.

### [Pharmaceutical Composition]

A fourth aspect of the present disclosure is a pharmaceutical composition. The pharmaceutical composition according to the present aspect contains the protein folding agent according to the first aspect, and a pharmaceutically acceptable carrier.

The pharmaceutical composition according to the present aspect can be used for treatment or prevention of a disease caused by aggregation of proteins. It is known that there is a risk that transition metal ions such as copper ions and nickel ions cause protein aggregation in vivo, leading to diseases. The pharmaceutical composition according to the present aspect contains the protein folding agent according to the first aspect as an active ingredient. The above-described protein folding agent suppresses aggregation of proteins in the presence of a transition metal, and promotes folding into a native structural protein. Therefore, it is possible to prevent the onset of the disease caused by aggregation of proteins, suppress the progression of the disease, and/or improve symptoms caused by the disease.

Examples of the disease caused by aggregation of proteins include folding diseases. The folding disease is a disease caused by aggregation and/or deposition of misfolded proteins. Examples of the folding disease include, but are not limited to, neurodegenerative diseases such as amyotrophic lateral sclerosis (ALS), Parkinson's disease, Alzheimer's disease, dementia with Lewy bodies, spinocerebellar degeneration, frontotemporal dementia, Pick's disease, Creutzfeldt-Jakob disease, and familial amyloid polyneuropathy; and type 2 diabetes.

A subject to which the pharmaceutical composition is administered is not particularly limited, and the pharmaceutical composition can be used for animals that develop the disease caused by aggregation of proteins. The subject to which the pharmaceutical composition is administered is preferably a mammal, and may be a human or a non-human mammal. Examples of the non-human mammal include, but are not limited to, primates (monkeys, chimpanzees, gorillas, and the like), rodents (mice, hamsters, rats, and the like), rabbits, dogs, cats, cows, goats, sheep, and horses.

The “pharmaceutically acceptable carrier” means a carrier that does not inhibit the physiological activity of the active ingredient and does not exhibit substantial toxicity to the administration subject. The phrase “does not exhibit substantial toxicity” means that the component does not exhibit toxicity to the administration subject at a dosage usually used. In the pharmaceutical composition according to the present embodiment, the pharmaceutically acceptable carrier is a carrier that does not inhibit the protein folding promoting effect of the protein folding agent according to the first aspect and does not exhibit substantial toxicity to the administration subject. The pharmaceutically acceptable carrier encompasses any known pharmaceutically acceptable component that is typically considered as an inactive ingredient. Examples of the pharmaceutically acceptable carrier include, but are not limited to, solvents, diluents, vehicles, excipients, glidants, binders, granulating agents, dispersing agents, suspending agents, wetting agents, lubricants, disintegrants, solubilizers, stabilizers, emulsifiers, and fillers. One type of the pharmaceutically acceptable carrier may be used alone, or two or more types thereof may be used in combination.

The pharmaceutical composition may contain other components in addition to the above-described components. The other components are not particularly limited, and those commonly used in the pharmaceutical field can be used without particular limitation. Examples of the other components include pharmaceutical additives other than those described above. Examples of the pharmaceutical additive include, but are not limited to, preservatives (for example, antioxidants), chelating agents, flavoring agents, sweeteners, thickeners, buffers, and coloring agents. The pharmaceutical composition may contain an active ingredient other than the protein folding agent according to the first aspect. Examples of the active ingredient include, but are not limited to, antiviral agents, antibiotics, anti-inflammatory agents, antipyretics, and analgesics.

The dosage form of the pharmaceutical composition is not particularly limited, and may be a dosage form generally used as a pharmaceutical preparation. The pharmaceutical composition according to the present embodiment may be an oral preparation or a parenteral preparation, but is preferably a parenteral preparation. Examples of the oral preparation include tablets, coated tablets, pills, powders, granules, capsules, syrups, fine granules, liquids, drops, and emulsions. Examples of the parenteral preparation include injections, suppositories, nasal drops, enteral agents, and inhalants. The pharmaceutical compositions of these dosage forms can be formulated according to a conventional method (for example, a method described in the Japanese Pharmacopoeia).

The administration route of the pharmaceutical composition according to the present embodiment is not particularly limited, and the pharmaceutical composition can be administered by an oral or parenteral route; but parenteral administration is preferable. Examples of the administration route for parenteral administration include intravenous administration, intranasal administration, subcutaneous administration, intradermal administration, intramuscular administration, intraperitoneal administration, and enteral administration.

The pharmaceutical composition can be administered in a therapeutically effective amount of the protein folding agent according to the first aspect. The “therapeutically effective amount” means an amount of a drug effective for treatment or prevention of a target disease. For example, the therapeutically effective amount of the protein folding agent described above can be an amount effective for refolding of aggregated proteins. The therapeutically effective amount may be appropriately determined depending on symptoms, body weight, age, and sex of a patient, a dosage form of the pharmaceutical composition, an administration method, and the like. For example, the pharmaceutical composition can be administered in a single dose of 0.01 to 1000 mg per 1 kg of body weight of the administration subject, as the antiviral agent. The above-described dose may be 0.05 to 500 mg/kg, 0.1 to 300 mg/kg, 0.2 to 200 mg/kg, or 0.3 to 100 mg/kg.

The pharmaceutical composition may contain a therapeutically effective amount of the above-described protein folding agent per unit dosage form. For example, a contained amount of the above-described protein folding agent in the pharmaceutical composition may be 0.01% to 90% by mass, 0.05% to 80% by mass, or 0.1% to 60% by mass.

The pharmaceutical composition may be administered once or repeatedly. In the case of repeated administration, the administration interval may be appropriately determined depending on symptoms, body weight, age, and sex of a patient, a dosage form of the pharmaceutical composition, an administration method, and the like. The administration interval can be, for example, every few hours, 2 to 3 times a day, once a day, once every 2 to 3 days, once a week, once a month, once every few months, or the like.

[Agent for suppressing cytotoxicity by transition metal]

A fourth aspect of the present disclosure is an agent for suppressing cytotoxicity by a transition metal. The above-described suppressing agent includes at least one or more compounds selected from the group consisting of the following (A) to (E):
(A) a cyclic polyamine that contains one or more thiol groups or disulfide bonds and is bindable to a transition metal ion (compound (A));
(B) a compound in which two or more of the cyclic polyamines (A) are bonded through a disulfide bond between the thiol groups (compound (B));
(C) a complex of the cyclic polyamine (A) or the compound (B) with a transition metal ion (the compound (C));
(D) a salt of the cyclic polyamine (A) or the compound (B) (compound (D)); and
(E) a solvate of the cyclic polyamine (A) or the compound (B) (compound (E))

The (A) to (D) are the same as (A) to (D) in the first aspect. The compounds (A) to (D) are the same as the compounds (A) to (D) described above.

Transition metal ions can have cytotoxicity. The compounds (A) to (E) can suppress the cytotoxicity caused by transition metal ions. Therefore, the compounds (A) to (E) can be used as an agent for suppressing cytotoxicity by a transition metal ion. The above-described agent for suppressing cytotoxicity preferably contains at least one selected from the group consisting of the compound (A), the compound (B), the compound (D), and the compound (E). The above-described agent for suppressing cytotoxicity preferably contains at least one selected from the group consisting of the compound (B) or the compound (A-2), a salt of the compound (B) or the compound (A-2), and a solvate of the compound (B) or the compound (A-2). The above-described agent for suppressing cytotoxicity may further contain at least one selected from the group consisting of the compound (A-1), a salt of the compound (A-1), and a solvate of the compound (A-1).

Examples of the transition metal ion include the same as those described above. Specific examples of the transition metal ion include, but are not limited to, copper ions (for example, Cu(II) ions and Cu(I) ions), nickel ions (for example, Ni(II) ions), iron ions (for example, Fe(II) ions and Fe(III) ions), manganese ions (for example, Mn(II) ions and Mn(III) ions), zinc ions (for example, Zn(II) ions), and molybdenum ions (for example, Mo (IV) ions and Mo (VI) ions).

The compound (A) and the compound (B) stably bind to the transition metal ion to form a complex with the transition metal ion. It is presumed that this can suppress cytotoxicity caused by the transition metal ion.

### EXAMPLES

Hereinafter, the present invention will be described with reference to Experimental Examples, but the present invention is not limited to Experimental Examples.

### Experimental Example 1

### Synthesis Examples of Compound (A-1-1-1) and Compound (B-1-1-1)

The compound (A-1-1-1) and the compound (B-1-1-1) were synthesized according to the following synthesis scheme. Numerical values in % in the synthesis scheme represent yields in each reaction.

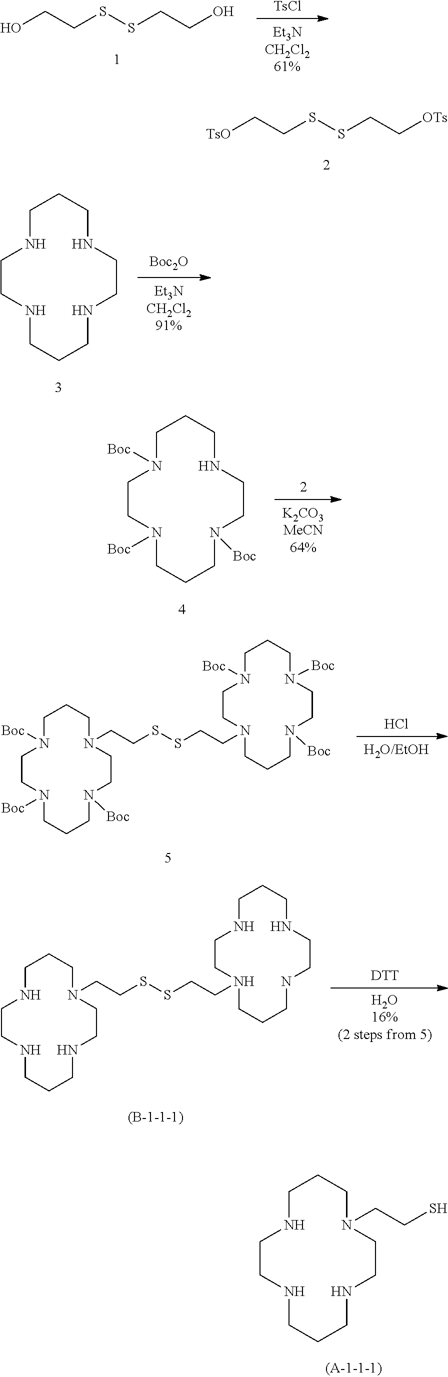

### (Synthesis of Compound 2 and Compound 4)

The compound 2 and the compound 4 were synthesized according to previous reports (P. Jain et al., Langmuir 2019, 35, 1864; B. Boitrel et al., Tetrahedron Lett. 1995, 36, 4995). In the above reaction formula, TsCl represents p-toluenesulfonyl chloride, Et₃N represents triethylamine, Boc₂O represents di-tert-butyl dicarbonate, Ts represents a p-toluenesulfonyl group, and Boc represents a tert-butoxycarbonyl group. The compound 1 is 2-hydroxyethyl disulfide, and the compound 3 is 1,4,8,11-tetraazacyclotetradecane (cyclam).

### (Synthesis of Compound 5)

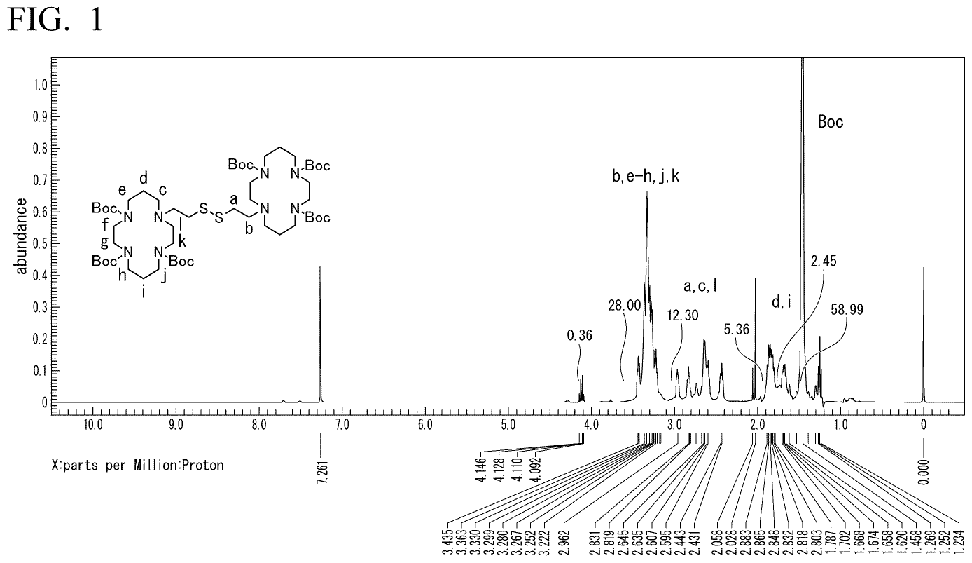

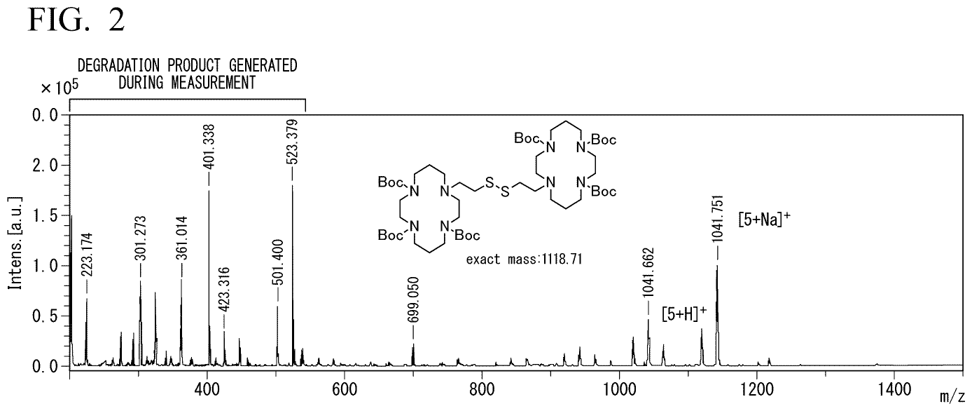

The compound 2 (139 mg, 0.300 mmol) was dissolved in 1.5 mL of dehydrated acetonitrile (MeCN), and mixed with potassium carbonate (103 mg, 0.747 mmol, 2.5 eq). The compound 4 (315 mg, 0.629 mmol, 2.1 eq) dissolved in 1.5 mL of dehydrated acetonitrile was added thereto, and the mixture was stirred at 60° C. under a nitrogen atmosphere. At 44 hours from the start of the reaction, the compound 4 (30 mg, 0.060 mmol, 0.2 eq) was added thereto again, and the mixture was stirred for 24 hours. The obtained suspension was suction-filtered, and the filtrate was dried under reduced pressure and purified by silica gel chromatography to obtain a compound 5 (214 mg, 0.192 mmol, 64%). The obtained compound was identified by 1H NMR measurement (400 MHz, CDCl₃, 323 K; FIG. 1) and MALDI-TOF mass spectrometric spectrum measurement (FIG. 2).

### (Synthesis of Compound (B-1-1-1))

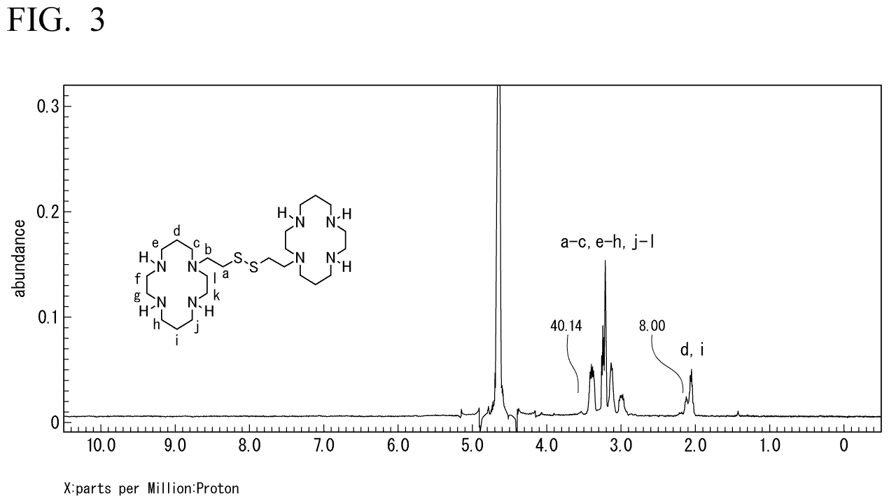

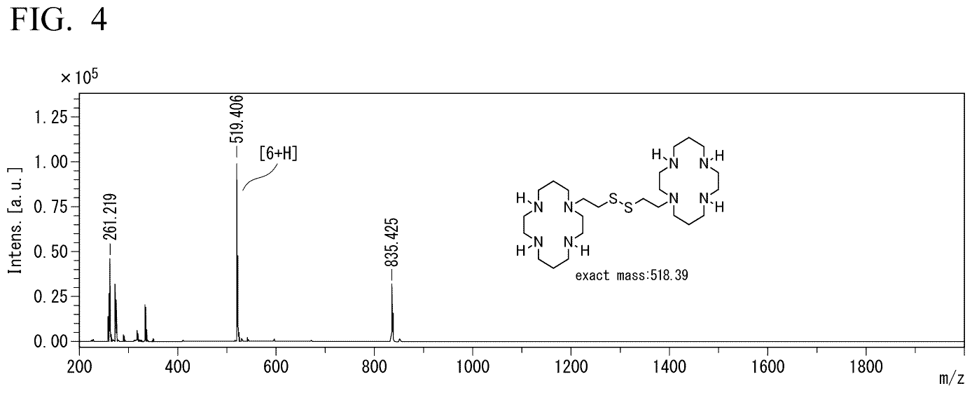

280 μL of concentrated hydrochloric acid was added to an ethanol solution (H₂O/EtOH) (280 μL) of the compound 5 (60 mg, 0.054 mmol) under ice-cooling, and the mixture was stirred for 10 minutes and then stirred at room temperature for 3 hours. The obtained white suspension was ice-cooled and suction-filtered to obtain a white solid. Furthermore, purification was carried out by reverse-phase HPLC to obtain a compound (B-1-1-1) (12 mg, 0.023 mmol, 43%). The obtained compound was identified by ¹H NMR measurement (400 MHz, D20, 323 K; FIG. 3) and MALDI-TOF mass spectrometric spectrum measurement (FIG. 4).

### (Synthesis of Compound (A-1-1-1))

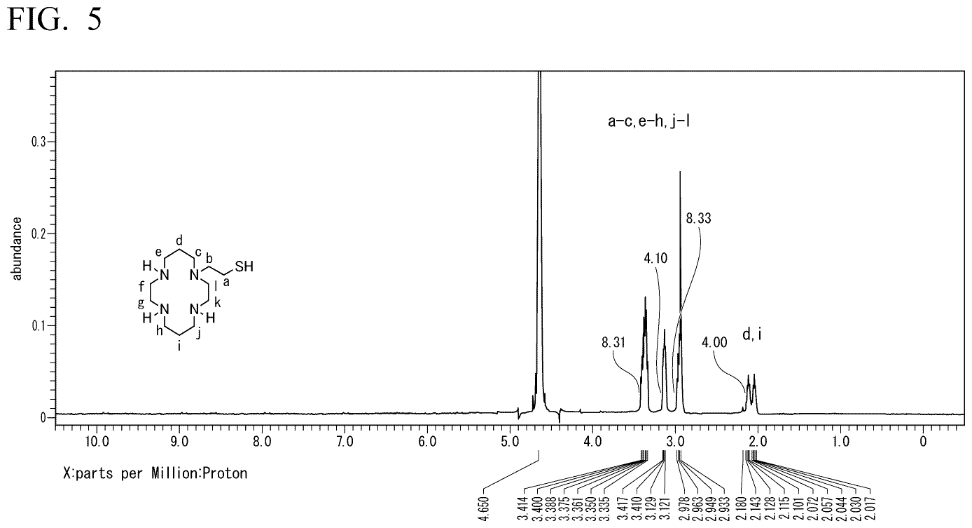

700 μL of concentrated hydrochloric acid was added to an ethanol solution (700 μL) of the compound 5 (150 mg, 0.134 mmol), and the mixture was stirred at room temperature for 15 hours. The obtained white suspension was ice-cooled and suction-filtered to obtain a white solid. Subsequently, 1,4-dithiothreitol (DTT) (19 mg, 0.12 mmol, 0.9 eq) and 2 mL of ultrapure water were added thereto, and the mixture was stirred at room temperature for 15 hours. Purification was carried out by reverse-phase HPLC to obtain a compound (A-1-1-1) (11 mg, 16% from compound 5). The obtained compound was identified by 1H NMR measurement (400 MHz, D20, 323 K; FIG. 5) and MALDI-TOF mass spectrometric spectrum measurement (FIG. 6).

### (Reagent)

2-Hydroxyethyl disulfide was purchased from Sigma-Aldrich Co. LLC (St. Louis, MO, USA). p-Toluenesulfonyl chloride was purchased from Kishida Chemical Co., Ltd. (Tokyo, Japan). Dichloromethane, dehydrated acetonitrile, potassium carbonate, and di-tert-butyl dicarbonate were purchased from Kanto Chemical Co., Inc. (Tokyo, Japan). 1,4,8,11-Tetraazacyclotetradecane (cyclam) was purchased from Tokyo Chemical Industry Co., Ltd. (Tokyo, Japan). Triethylamine and hydrochloric acid were purchased from Fujifilm Wako Pure Chemical Corporation (Osaka, Japan). 1,4-Dithiothreitol was purchased from Nacalai Tesque, Inc. (Kyoto, Japan).

### (Device)

[1] For the NMR measurement, JNM-ECX400 device manufactured by JEOL Ltd. was used. The MALDI-TOF mass spectrometric spectrum measurement was carried out using an autoflex speed device manufactured by Bruker Corporation. The HPLC purification was carried out using a PU-4086-Binary pump system and a UV-4075 detector manufactured by JASCO Corporation, a YMC-Actus Triart C18 column (250×20.0 mm) manufactured by YMC Co., Ltd., and a CHF122SC fraction collector manufactured by Advantec Toyo Kaisha, Ltd., at a flow rate of 18.9 mL/min.

### <Protein Folding Test>

Bovine Pancreatic Trypsin Inhibitor (BPTI) having three disulfide bonds in the molecule was used as a model protein for analyzing a folding process.

### (Quantitative Determination of Concentration of Compound (A-1-1-1))

A solution of the compound (A-1-1-1) dissolved in a 10 mM hydrochloric acid aqueous solution and a 10 mM Ellman's reagent (5,5′-dithio-bis-(2-nitrobenzoic acid)) were mixed in a 50 mM Tris-HCl buffer (pH: 7.5, 0.3 M NaCl), and UV absorption measurement was carried out at 30° C. The concentration of the compound (A-1-1-1) solution was determined from an absorbance at 412 nm.

### (Preparation of Reduced and Denatured BPTI)

BPTI (10 mg) was dissolved in 0.1 M Tris-HCl (pH: 8.0, 1.0 mL) containing 30 mM 1,4-dithiothreitol and 8 M urea, incubated at 50° C. for 3 hours, and purified by reverse-phase HPLC. HPLC purification was carried out using a PU-4180 pump and a UV-4075 detector manufactured by JASCO Corporation (Tokyo, Japan), and an InertSustain C18 column (4.6 mm×250 mm) manufactured by GL Sciences Inc. (Tokyo, Japan) at a flow rate of 1.0 mL·min⁻¹. The collected fractions were freeze-dried, and the obtained powder was stored at −30° C.

### (Reagent)

BPTI was purchased from Pro-Spec-Tany TechnoGene Ltd. (Rehovot, Israel). 1,4-Dithiothreitol and urea were purchased from Nacalai Tesque, Inc. (Kyoto, Japan).

### (Device)

For analysis of the folding process by reverse-phase HPLC, Primaide HPLC system manufactured by Hitachi High-Tech Corporation (Tokyo, Japan) and TSKgel Protein C4-300 column (§ 4.6×150 mm) manufactured by Tosoh Corporation (Tokyo, Japan) were used.

### (Protein Folding Test Using Glutathione)

In the presence of 200 μM oxidized glutathione (GSSG) and 1.0 mM reduced glutathione (GSH), reduced and denatured BPTI (30 μM) was dissolved in a Tris-HCl buffer (50 mM Tris-HCl, 300 mM NaCl, pH: 7.5) and incubated at 30° C. Reaction solutions (200 μL) collected at 0 minutes, 1 minute, 5 minutes, 10 minutes, 30 minutes, and 60 minutes after the start of the reaction were mixed with an equivalent amount of 1 M hydrochloric acid, and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

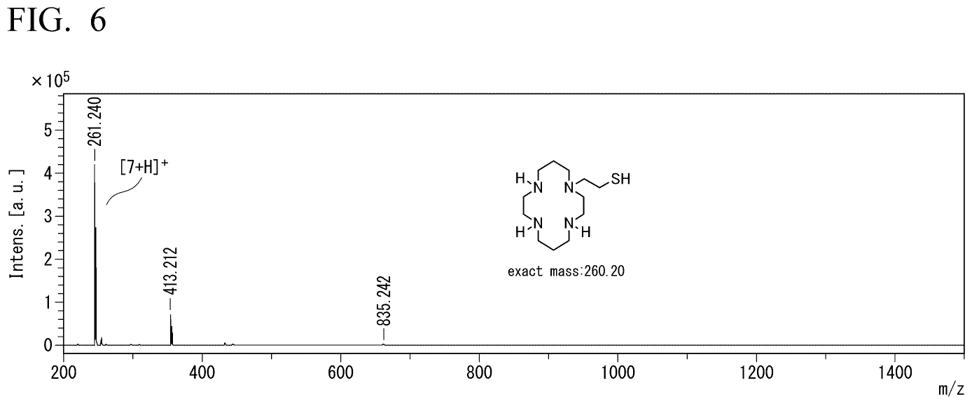

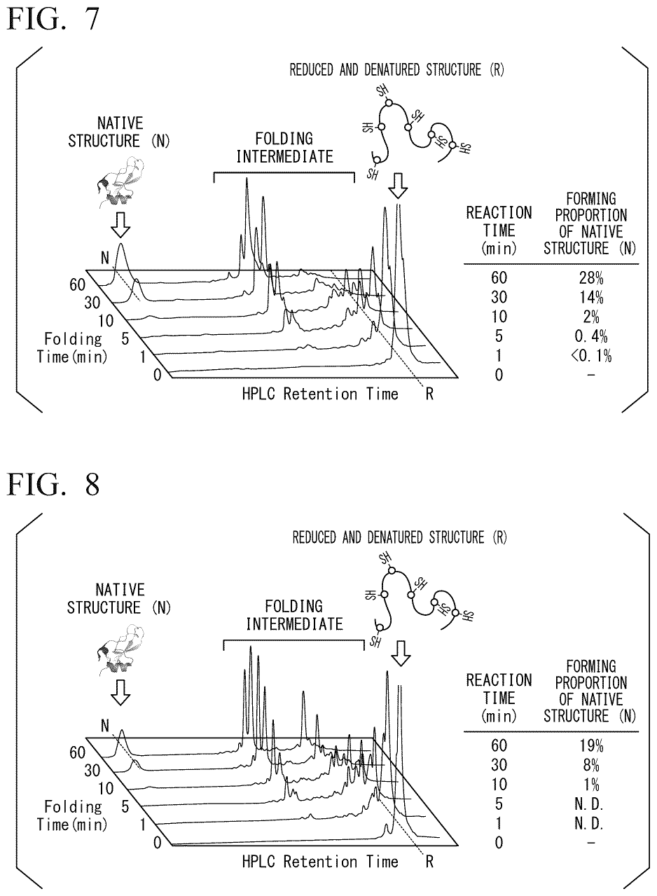

The results are shown in FIG. 7. It was confirmed that the reduced and denatured BPTI was folded over time to form a native structural BPTI.

### (Protein Folding Test Using Compound (A-1-1-1) and Compound (B-1-1-1))

In the presence of 200 μM of the compound (B-1-1-1) and 1.0 mM of the compound (A-1-1-1), reduced and denatured BPTI (30 μM) was dissolved in a Tris-HCl buffer (50 mM Tris-HCl, 300 mM NaCl, pH: 7.5) and incubated at 30° C. Reaction solutions (200 μL) collected at 0 minutes, 1 minute, 5 minutes, 10 minutes, 30 minutes, and 60 minutes after the start of the reaction were mixed with an equivalent amount of 1 M hydrochloric acid, and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

The results are shown in FIG. 8. It was confirmed that the reduced and denatured BPTI was folded over time to form a native structural BPTI.

### (Protein Folding Test in Presence of Transition Metal Ion Using Compound (A-1-1-1) and Compound (B-1-1-1))

In the presence of 200 μM of the compound (B-1-1-1) and 1.0 mM of the compound (A-1-1-1), 120 μM of a metal salt (NiCl₂ or CuCl₂) and reduced and denatured BPTI (30 μM) were dissolved in a Tris-HCl buffer (50 mM Tris-HCl, 300 mM NaCl, pH: 7.5) and incubated at 30° C. The reaction solution (200 μL) 1 hour after the start of the reaction was mixed with an equivalent amount of 1 M hydrochloric acid and analyzed by reverse-phase HPLC. Tracing was performed at a flow rate of 1.0 mL min 1 and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

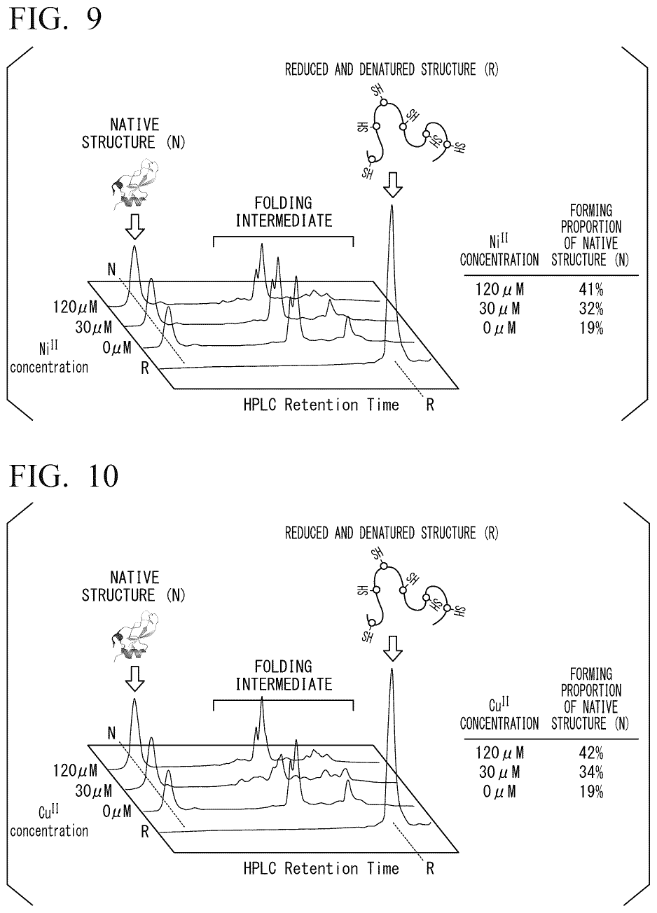

The results of the folding test in the presence of nickel (II) ions (Ni²⁺) are shown in FIG. 9. It was confirmed that, as the concentration of nickel (II) ion increased, a formation proportion of the native structural BPTI increased, and the folding was promoted.

The results of the folding test in the presence of copper (II) ions (Cu²⁺) are shown in FIG. 10. It was confirmed that, as the concentration of copper (II) ion increased, a formation proportion of the native structural BPTI increased, and the folding was promoted.

From these results, it was found that the compound (A-1-1-1) and the compound (B-1-1-1) promoted the folding of the reduced and denatured protein in the presence of a transition metal ion.

### (Protein Folding Test Under Various Conditions)

Reduced and denatured BPTI (30 μM) and the following compounds were dissolved in a Tris-HCl buffer (50 mM Tris-HCl, 300 mM NaCl, pH: 7.5) to prepare each reaction solution of (1) to (5).
(1) GSSG (200 μM), GSH (1.0 mM), and CuCl₂ (120 μM)
(2) GSSG (200 μM), GSH (1.0 mM), cyclam (1.4 mM), and CuCl₂ (120 μM)
(3) GSSG (200 μM) and GSH (1.0 mM)
(4) Compound (A-1-1-1) (1.0 mM), Compound (B-1-1-1) (200 μM), and CuCl₂ (120 μM)
(5) Compound (A-1-1-1) (1.0 mM) and Compound (B-1-1-1) (200 μM)

The above reaction solutions (1) to (5) were incubated at 30° C. The reaction solution (200 μL) 1 hour after the start of the reaction was mixed with an equivalent amount of 1 M hydrochloric acid and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

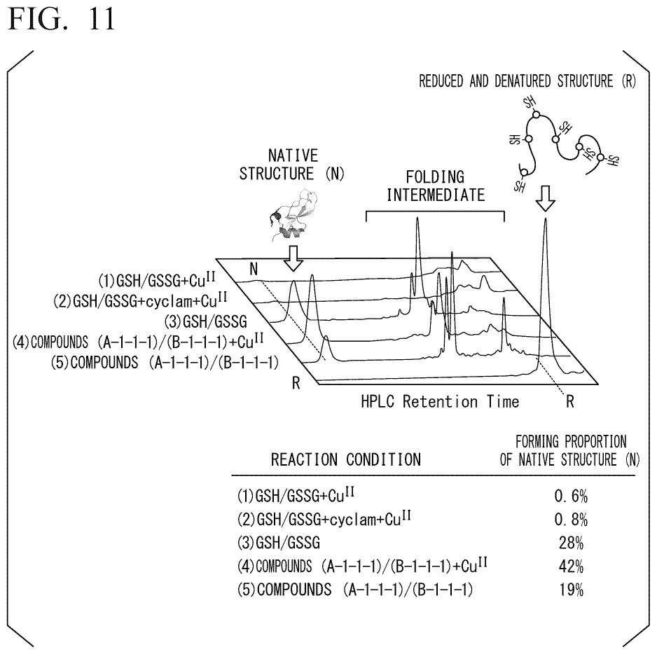

The results are shown in FIG. 11. When glutathione (GSH/GSSH) was used, the folding did not proceed in the presence of copper ions, and native structural BPTI was barely formed (reaction solution (1)). Even when cyclam was added to glutathione (GSH/GSSH), the folding did not proceed in the presence of copper ions, and native structural BPTI was barely formed (reaction solution (2)).

On the other hand, when the compound (A-1-1-1) and the compound (B-1-1-1) were used, the folding was further promoted in the presence of copper ions as compared with the absence of copper ions (reaction solutions (3) and (4)). From these results, it was found that the compound (A-1-1-1) and the compound (B-1-1-1) promoted the folding of the reduced and denatured protein in the presence of a transition metal ion.

### (Protein Folding Test in Presence of Zn(II) Ions Using Compound (A-1-1-1) and Compound (B-1-1-1))

In the presence of 200 μM oxidized glutathione (GSSG) and 1.0 mM reduced glutathione (GSH), or in the presence of 200 μM of the compound (B-1-1-1) and 1.0 mM of the compound (A-1-1-1), ZnSO₄ and reduced and denatured BPTI (30 μM) were dissolved in a Tris-HCl buffer (50 mM Tris-HCl, 300 mM NaCl, pH: 7.5) and incubated at 30° C. The reaction solution (200 μL) 1 hour after the start of the reaction was mixed with an equivalent amount of 1 M hydrochloric acid and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

The results are shown in FIG. 12. When GSSG and GSH were used, the formation proportion of native structural BPTI was less than 1% in the presence of Zn(II) ions. This result indicates that the protein folding promotion effect by GSSG and GSH is significantly inhibited by Zn(II) ions. On the other hand, when the compound (A-1-1-1) and the compound (B-1-1-1) were used, the formation proportion of native structural BPTI was improved in the presence of Zn(II) ions as compared with the absence of Zn(II) ions. This result indicates that the protein folding promotion effect of the compound (A-1-1-1) and the compound (B-1-1-1) is promoted by Zn(II) ions.

### <Cytotoxicity Evaluation Test>

### (Cytotoxicity Test of Compound (A-1-1-1) and Compound (B-1-1-1))

HeLa cells were seeded in a 96-well plate at 5,000 cells/well and cultured at 37° C. in a 5% CO₂ environment for 24 hours. Subsequently, a mixed solution (10:1) of a certain amount of the compound (A-1-1-1) and the compound (B-1-1-1) was added thereto. 24 hours later, Cell Counting Kit-8 (DOJINDO LABORATORIES) solution was added thereto. Two hours later, the absorbance at 450 nm was measured to estimate cell viability.

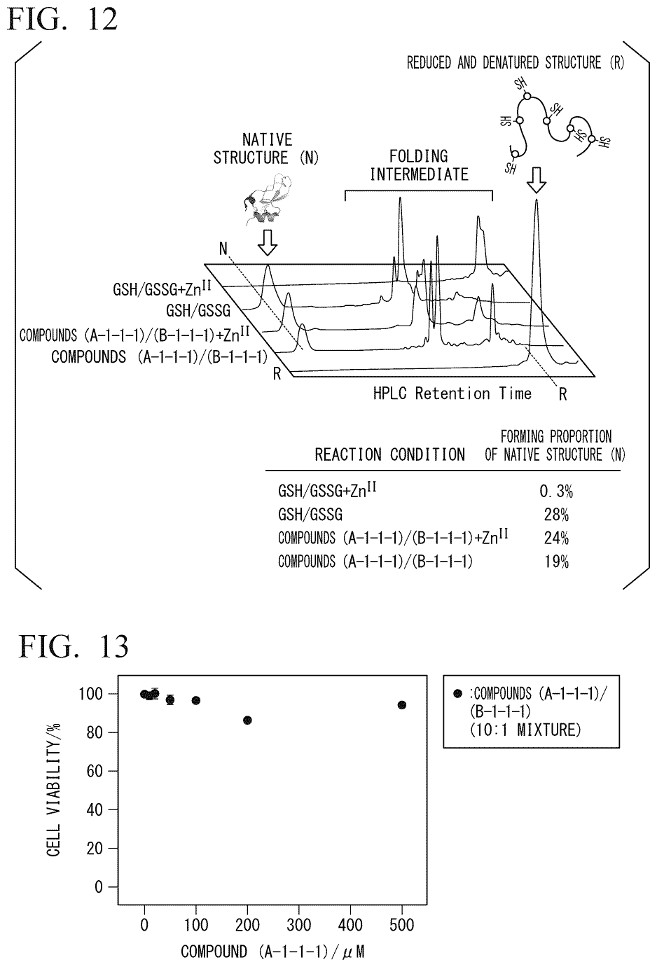

The results are shown in FIG. 13. The 10:1 mixture of the compounds (A-1-1-1) and (B-1-1-1) did not significantly affect the viability of HeLa cells in the tested concentration range. Based on these results, the compounds (A-1-1-1) and (B-1-1-1) were found to exhibit limited cytotoxicity.

### (Suppressive Effect of Compound (A-1-1-1) and Compound (B-1-1-1) on Cytotoxicity of Cu(II) Ions)

HeLa cells were seeded in a 96-well plate at 5,000 cells/well and cultured at 37° C. in a 5% CO₂ environment for 24 hours. Subsequently, a CuCl₂ aqueous solution was added thereto to obtain 100 μM Cu(II). Four hours later, a mixed solution (10:1) of a certain amount of the compound (A-1-1-1) and the compound (B-1-1-1), or a mixed solution (10:1) of oxidized glutathione (GSSG) and reduced glutathione (GSH) was added. 24 hours after the addition of CuCl₂, a Cell Counting Kit-8 solution was added thereto. Two hours later, an absorbance at 450 nm was measured to estimate the cell viability.

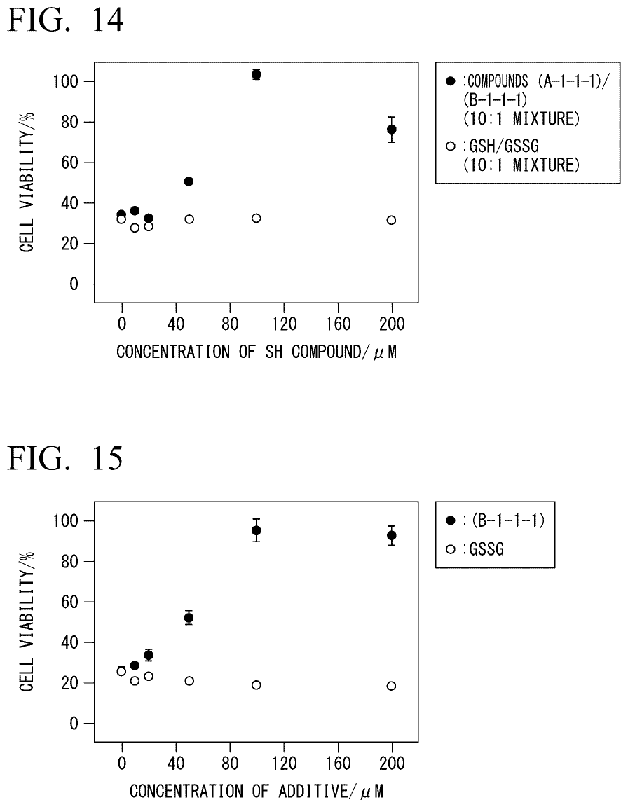

The results are shown in FIG. 14. By adding 100 μM of Cu(II) ions, the cell viability of HeLa cells decreased to approximately 30%. The addition of GSH and GSSG had little effect on improving the cell viability of HeLa cells. On the other hand, the addition of the compound (A-1-1-1) and the compound (B-1-1-1) greatly improved the viability of HeLa cells. Based on these results, the mixture of the compounds (A-1-1-1) and (B-1-1-1) was found to reduce cytotoxicity by Cu(II) ions.

### (Suppressive Effect of Compound (B-1-1-1) on Cytotoxicity of Cu(II) Ions)

HeLa cells were seeded in a 96-well plate at 5,000 cells/well and cultured at 37° C. in a 5% CO₂ environment for 24 hours. Subsequently, a CuCl₂ aqueous solution was added thereto to obtain 100 μM Cu(II). Four hours later, a certain amount of the compound (B-1-1-1) solution or oxidized glutathione (GSSG) solution was added thereto. 24 hours after the addition of CuCl₂, a Cell Counting Kit-8 solution was added thereto. Two hours later, the absorbance at 450 nm was measured to estimate the cell viability.

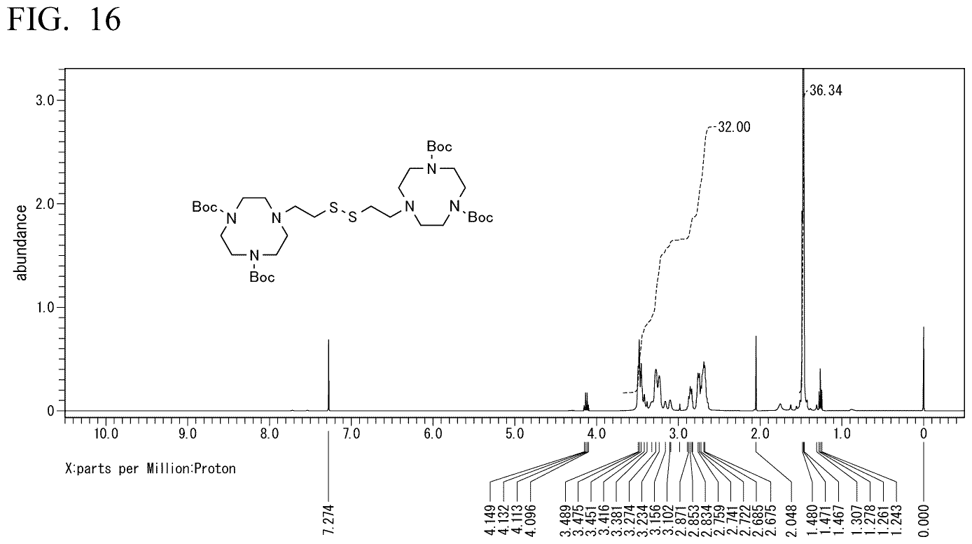

The results are shown in FIG. 15. By adding 100 μM of Cu(II) ions, the cell viability of HeLa cells decreased to approximately 20%. The addition of GSSG had little effect on improving the cell viability of HeLa cells. On the other hand, the addition of the compound (B-1-1-1) greatly improved the viability of HeLa cells. Based on these results, the compound (B-1-1-1) was found to reduce cytotoxicity by Cu(II) ions.

### Experimental Example 2

### Synthesis Examples of Compound (A-1-2-1) and Compound (B-1-2-1)

The compound (A-1-2-1) and the compound (B-1-2-1) were synthesized according to the following synthesis scheme. Numerical values in % in the synthesis scheme represent yields in each reaction.

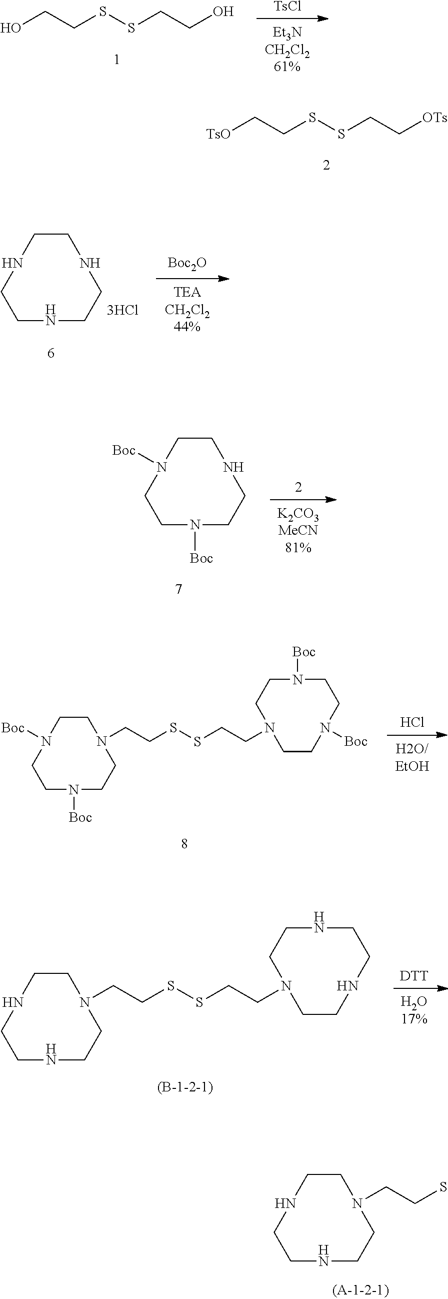

### (Synthesis of Compounds 2 and 7)

The compounds 2 and 7 were synthesized according to previous reports (P. Jain et al., Langmuir 2019, 35, 1864; M. Starck et al., Bioconjugate Chem. 2020, 31, 229). In the above reaction formula, TsCl represents p-toluenesulfonyl chloride, Et₃N represents triethylamine, Boc₂O represents di-tert-butyl dicarbonate, Ts represents a p-toluenesulfonyl group, and Boc represents a tert-butoxycarbonyl group. The compound 1 is 2-hydroxyethyl disulfide, and the compound 6 is 1,4,7-triazacyclononane trihydrochloride (TACN·3HCl).

### (Synthesis of Compound 8)

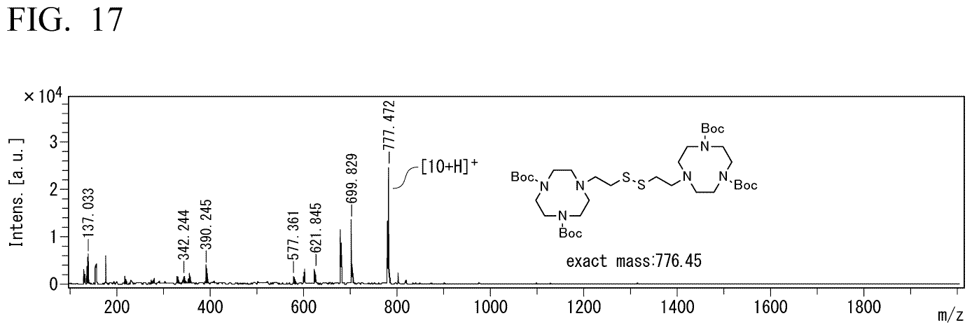

The compound 8 (245 mg, 0.745 mmol) was dissolved in 3.5 mL of dehydrated acetonitrile and mixed with potassium carbonate (103 mg, 0.747 mmol, 2.5 eq). The compound 2 (117 mg, 0.849 mmol, 2.5 eq) was added thereto, and the mixture was stirred at 60° C. for 39 hours under a nitrogen atmosphere. The obtained suspension was suction-filtered, and the filtrate was dried under reduced pressure and purified by silica gel chromatography to obtain a compound 8 (215 mg, 0.276 mmol, 181%). The obtained compound 8 was identified by 1H NMR measurement (FIG. 16) and MALDI-TOF mass spectrometric spectrum measurement (FIG. 17).

### (Synthesis of Compound (B-1-2-1))

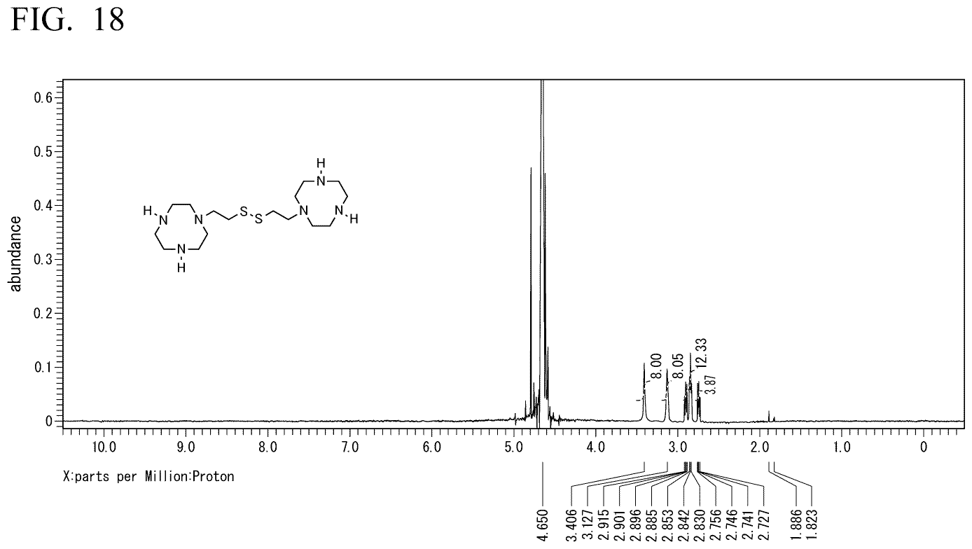

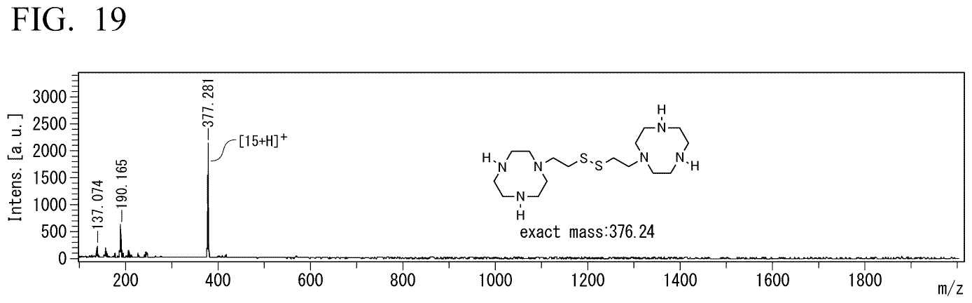

320 μL of concentrated hydrochloric acid was added to an ethanol solution (320 μL) of the compound 8 (44 mg, 0.057 mmol) under ice-cooling, and the mixture was stirred for 10 minutes and then stirred at room temperature for 3 hours. The obtained white suspension was ice-cooled and suction-filtered to obtain a white solid. Furthermore, purification was carried out by reverse-phase HPLC to obtain a compound (B-1-2-1) (4.5 mg, 0.012 mmol, 21%). The obtained compound (B-1-2-1) was identified by 1H NMR measurement (FIG. 18) and MALDI-TOF mass spectrometric spectrum measurement (FIG. 19).

### (Synthesis of Compound (A-1-2-1))

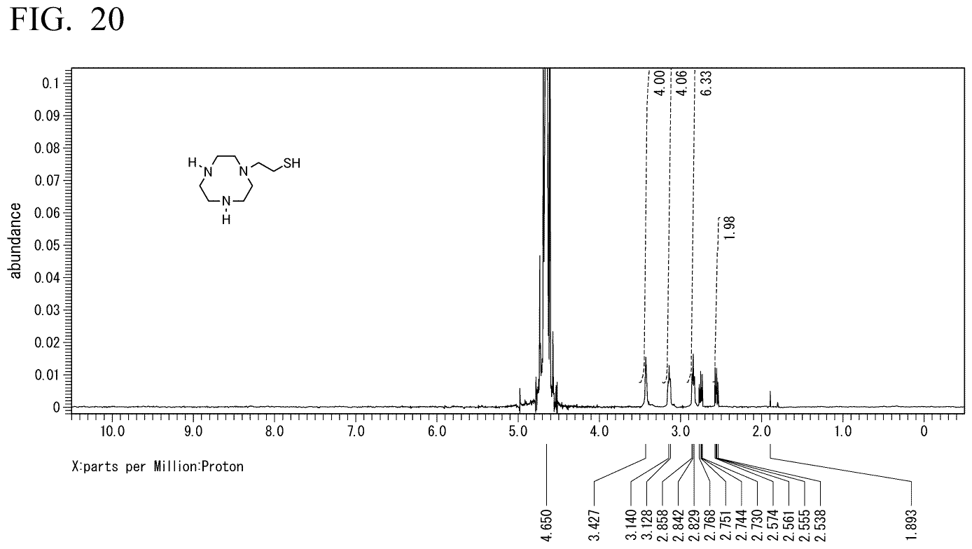

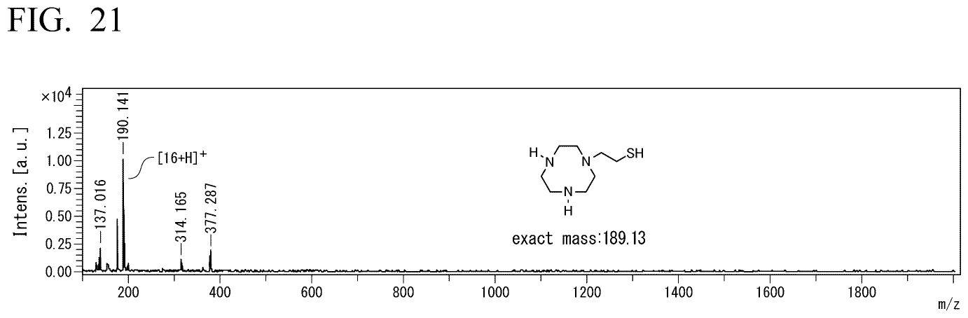

320 μL of concentrated hydrochloric acid was added to an ethanol solution (320 μL) of the compound 8 (44 mg, 0.057 mmol), and the mixture was stirred for 10 minutes and then stirred at room temperature for 3 hours. The obtained white suspension was ice-cooled and suction-filtered to obtain a white solid. Subsequently, 1,4-dithiothreitol (41 mg, 0.26 mmol, 10 eq) and 1.1 mL of ultrapure water were added thereto, and the mixture was stirred at room temperature for 45 hours. Purification was carried out by reverse-phase HPLC to obtain a compound (A-1-2-1) (3.6 mg, 0.0192 mmol, 17% from compound 8). The obtained compound (A-1-2-1) was identified by 1H NMR measurement (FIG. 20) and MALDI-TOF mass spectrometric spectrum measurement (FIG. 21).

### <Protein Folding Test>

BPTI having three disulfide bonds in the molecule was used as a model protein for analyzing a folding process.

### (Quantitative Determination of Concentration of Compound (A-1-2-1))

A solution of the compound (A-1-2-1) dissolved in a 10 mM hydrochloric acid aqueous solution and a 10 mM Ellman's reagent (5,5′-dithio-bis-(2-nitrobenzoic acid)) were mixed in a 50 mM Tris-HCl buffer (pH: 7.5, 0.3 M NaCl), and UV absorption measurement was carried out at 30° C. The concentration of the compound (A-1-2-1) solution was determined from an absorbance at 412 nm.

### (Preparation of Reduced and Denatured BPTI)

Reduced and denatured BPTI was prepared in the same manner as in Experimental Example 1.

### (Protein Folding Test Using Compound (A-1-2-1) and Compound (B-1-2-1))

In the presence of 90 μM of the compound (B-1-2-1) and 900 μM of the compound (A-1-2-1), or in the presence of 90 μM of oxidized glutathione (GSH) and 900 μM of reduced glutathione (GSSG), reduced and denatured BPTI (30 μM) was dissolved in a Tris-HCl buffer (50 mM Tris-HCl, 300 mM NaCl, pH: 7.5) and incubated at 30° C. The reaction solution (200 μL) 1 hour after the start of the reaction was mixed with an equivalent amount of 1 M hydrochloric acid and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

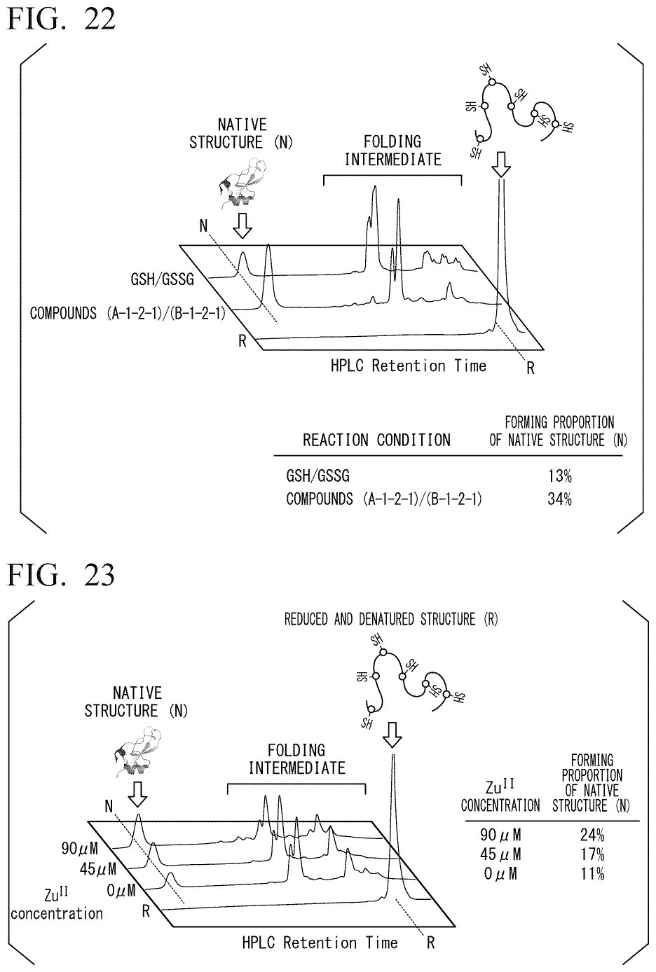

The results are shown in FIG. 22. The compound (A-1-2-1) and the compound (B-1-2-1) showed a higher formation proportion of native structural BPTI compared to GSH and GSSG. Based on these results, the compounds (A-1-2-1) and (B-1-2-1) were found to promote protein folding more effectively than GSH and GSSG.

### (Protein Folding Test in Presence of Zn(II) Ions Using Compound (B-1-2-1))

In the presence of 90 μM of the compound (B-1-2-1), 90 μM of Zn(II) ion (ZnSO₄) and reduced and denatured BPTI (30 μM) were dissolved in a Tris-HCl buffer (50 mM Tris-HCl, 300 mM NaCl, pH: 7.5) and incubated at 30° C. The reaction solution (200 μL) 1 hour after the start of the reaction was mixed with an equivalent amount of 1 M hydrochloric acid and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

The results are shown in FIG. 23. In the presence of the compound (B-1-2-1), the formation proportion of native structural BPTI increased as the Zn(II) ion concentration increased. Based on these results, it was found that the protein folding promotion effect of the compound (B-1-2-1) increased as the Zn(II) ion concentration increased. In addition, since the native structural BPTI was formed even at a Zn(II) ion concentration of 0 μM, it was found that the compound (B-1-2-1) had a protein folding promoting effect even in the absence of Zn(II) ions.

(Protein folding test in presence of Zn(II) ions using compound (A-1-2-1) and compound (B-1-2-1))

In the presence of 90 μM of the compound (B-1-2-1) and 900 μM of the compound (A-1-2-1), or in the presence of 90 μM of oxidized glutathione (GSH) and 900 μM of reduced glutathione (GSSG), 180 μM of Zn(II) ion (ZnSO₄) and reduced and denatured BPTI (30 μM) were dissolved in a Tris-HCl buffer (50 mM Tris-HCl, 300 mM NaCl, pH: 7.5) and incubated at 30° C. The reaction solution (200 μL) 1 hour after the start of the reaction was mixed with an equivalent amount of 1 M hydrochloric acid and analyzed by reverse-phase HPLC. Tracing was performed at a flow rate of 1.0 mL min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

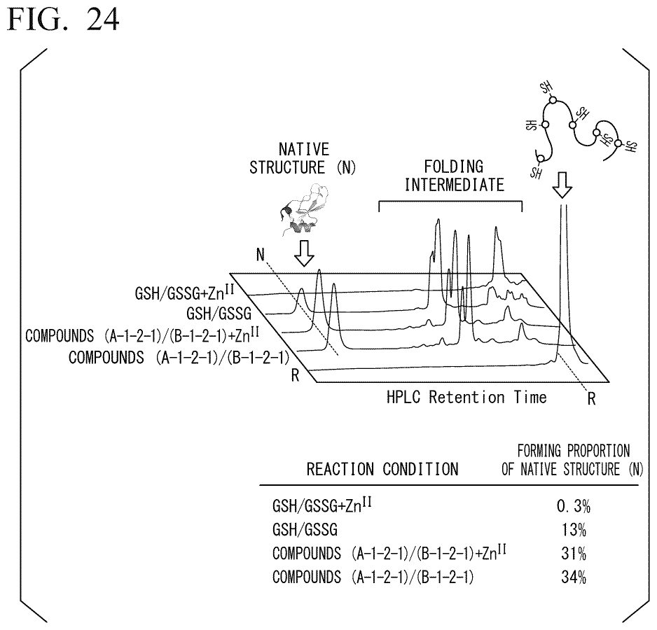

The results are shown in FIG. 24. In the presence of Zn(II) ions, when GSH and GSSG were used, proteins aggregated and folding hardly proceeded. On the other hand, when the compound (A-1-2-1) and the compound (B-1-2-1) were used, even in the presence of Zn(II) ions, native structural BPTI was formed to the same extent as in the absence of Zn(II) ions. Based on these results, it was found that that the compound (A-1-2-1) and the compound (B-1-2-1) had a protein folding promotion effect both in the presence and absence of Zn(II) ions.

### (Protein Folding Test in Presence of Zn(II) Ions Using Compound (B-1-2-1))

In the presence of 90 μM of the compound (B-1-2-1) or in the presence of 900 μM of reduced glutathione (GSSG), 90 μM of Zn(II) ion (ZnSO₄) and reduced and denatured BPTI (30 μM) were dissolved in a Tris-HCl buffer (50 mM Tris-HCl, 300 mM NaCl, pH: 7.5) and incubated at 30° C. The reaction solution (200 μL) 1 hour after the start of the reaction was mixed with an equivalent amount of 1 M hydrochloric acid and analyzed by reverse-phase HPLC. Tracing was performed at a flow rate of 1.0 mL min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

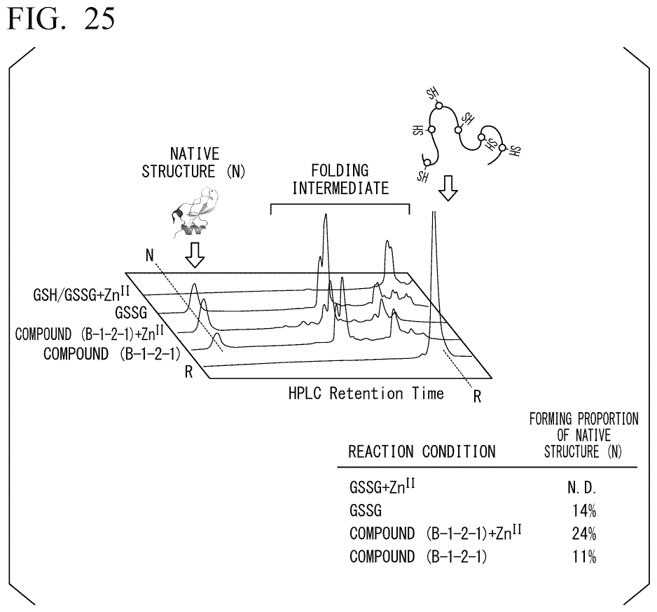

The results are shown in FIG. 25. In the presence of Zn(II) ions, when GSSG was used, proteins aggregated and folding hardly proceeded. On the other hand, when the compound (B-1-2-1) was used, the formation proportion of native structural BPTI was larger in the presence of Zn(II) ions than in the absence of Zn(II) ions. Based on these results, it was found that the compound (B-1-2-1) had a higher protein folding promotion effect in the presence of Zn(II) ions.

### Experimental Example 3

### Synthesis Example of Compound (B-2-1-1)

The compound (B-2-1-1) was synthesized according to the following synthesis scheme. Numerical values in % in the synthesis scheme represent yields in each reaction.

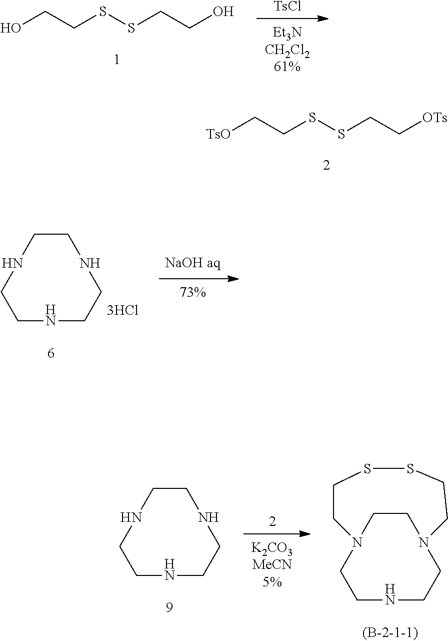

### (Synthesis of Compounds 2 and 9)

The compounds 2 and 9 were synthesized according to previous reports (P. Jain et al., Langmuir 2019, 35, 1864; C. A. Barta et al., Inorg Chem. 2008, 47, 2280). In the above reaction formula, MeCN represents acetonitrile.

### Synthesis Example of Compound (B-2-1-1)

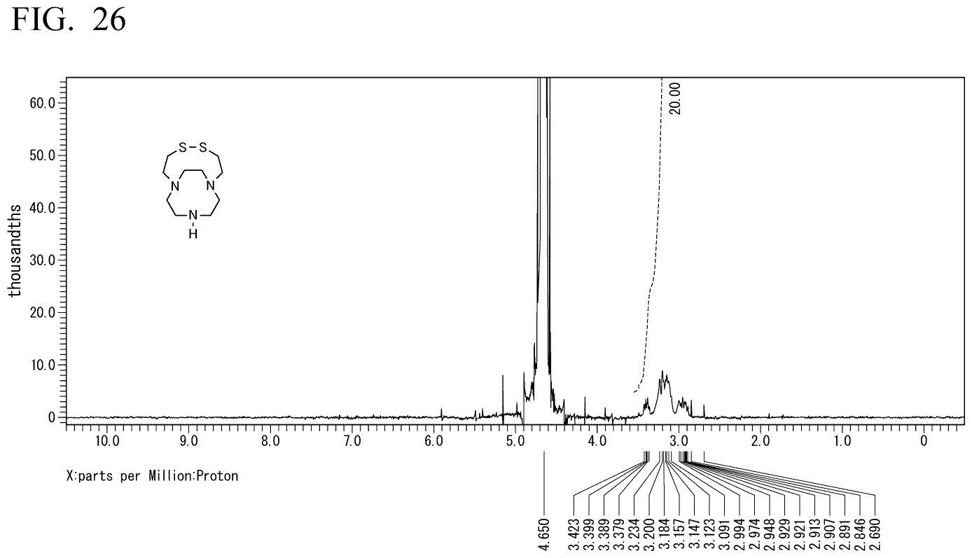

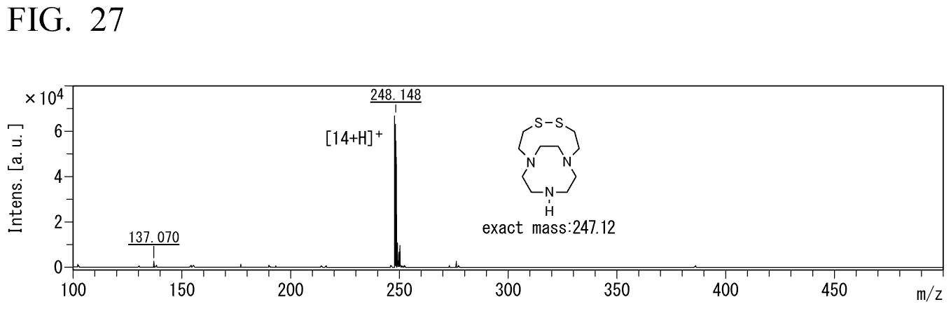

The compound 9 (14 mg, 0.11 mmol) was dissolved in 4 mL of dehydrated acetonitrile and mixed with potassium carbonate (38 mg, 0.28 mmol, 2.5 eq). The compound 2 (51 mg, 0.11 mmol, 1.0 eq) was added thereto, and the mixture was stirred at 60° C. for 24 hours under a nitrogen atmosphere. The obtained suspension was suction-filtered, and the filtrate was dried under reduced pressure to obtain a yellow solid. Furthermore, purification was carried out by reverse-phase HPLC to obtain a compound (B-2-1-1) (1.3 mg, 0.0052 mmol, 5%). The obtained compound was identified by 1H NMR measurement (FIG. 26) and MALDI-TOF mass spectrometric spectrum measurement (FIG. 27).

### <Protein Folding Test>

BPTI having three disulfide bonds in the molecule was used as a model protein for analyzing a folding process.

### (Preparation of Reduced and Denatured BPTI)

Reduced and denatured BPTI was prepared in the same manner as in Experimental Example 1.

### (Protein Folding Test Using Compound (B-2-1-1))

In the presence of 90 μM of the compound (B-2-1-1) or in the presence of 90 μM of oxidized glutathione (GSSG), reduced and denatured BPTI (30 μM) was dissolved in a Tris-HCl buffer (50 mM Tris-HCl, 300 mM NaCl, pH: 7.5) and incubated at 30° C. The reaction solution (200 μL) 1 hour after the start of the reaction was mixed with an equivalent amount of 1 M hydrochloric acid and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

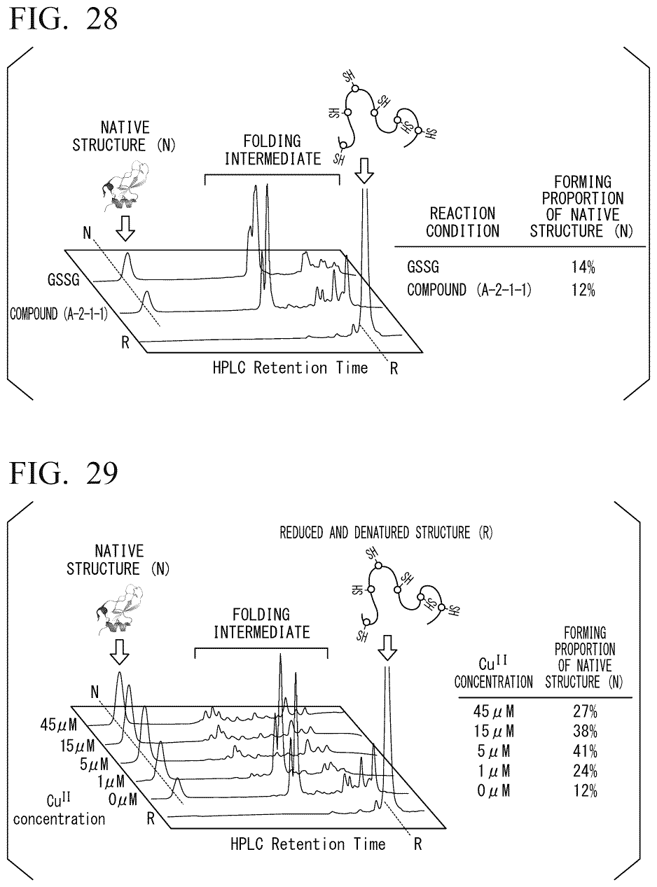

The results are shown in FIG. 28. The compound (B-2-1-1) exhibited a protein folding promotion effect comparable to that of GSSG.

### (Protein Folding Test in Presence of Transition Metal Ion Using Compound (B-2-1-1))

In the presence of 90 μM of the compound (B-2-1-1), 90 μM of Cu(II) ion (CuCl₂) or 90 μM of Ni(II) ion (NiCl₂) and reduced and denatured BPTI (30 μM) were dissolved in a Tris-HCl buffer (50 mM Tris-HCl, 300 mM NaCl, pH: 7.5) and incubated at 30° C. The reaction solution (200 μL) 1 hour after the start of the reaction was mixed with an equivalent amount of 1 M hydrochloric acid and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

The results in the presence of Cu(II) ions are shown in FIG. 29. In the presence of the compound (B-2-1-1), the formation proportion of native structural BPTI increased as the Cu(II) ion concentration increased. Based on these results, it was found that the protein folding promotion effect of the compound (B-2-1-1) increased as the Cu(II) ion concentration increased.

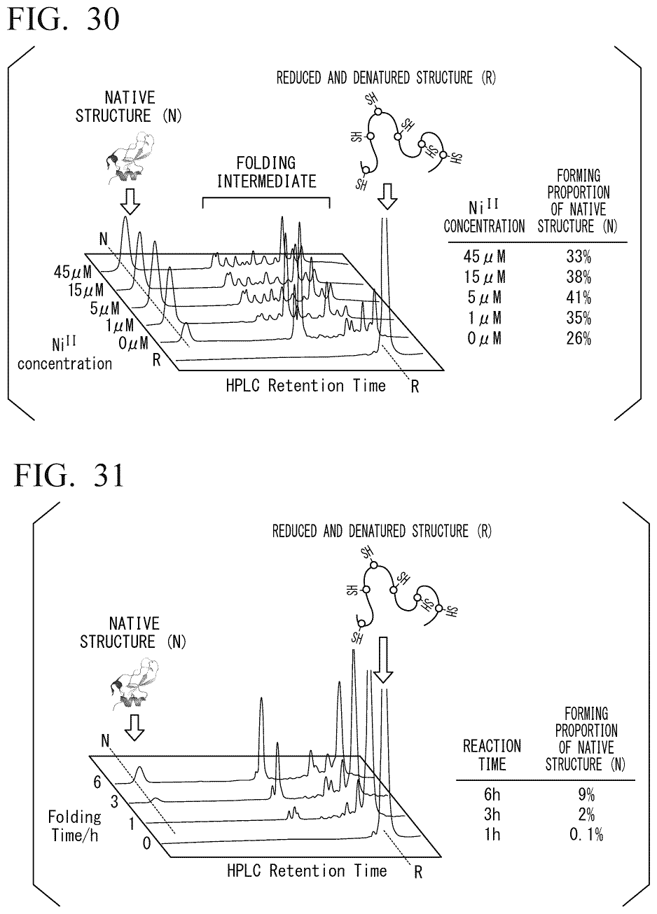

The results in the presence of Ni(II) ions are shown in FIG. 30. In the presence of the compound (B-2-1-1), the formation proportion of native structural BPTI increased as the Ni(II) ion concentration increased. Based on these results, it was found that the protein folding promotion effect of the compound (B-2-1-1) increased as the Ni(II) ion concentration increased.

### (Protein Folding Test in Presence of Ni(II) Ion Using a Catalytic Amount of Compound (B-2-1-1))

In the presence of 6 μM of the compound (B-2-1-1), NiCl₂ (0 μM or 1 μM) and reduced and denatured BPTI (30 μM) were dissolved in a Tris-HCl buffer (50 mM Tris-HCl, 300 mM NaCl, pH: 7.5) and incubated at 30° C. The reaction solution (200 μL) after a certain period of time from the start of the reaction was mixed with an equivalent amount of 1 M hydrochloric acid and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

The results in the absence of Ni(II) ions are shown in FIG. 31. It was found that even a catalytic amount of the compound (B-2-1-1) had the protein folding promotion effect.

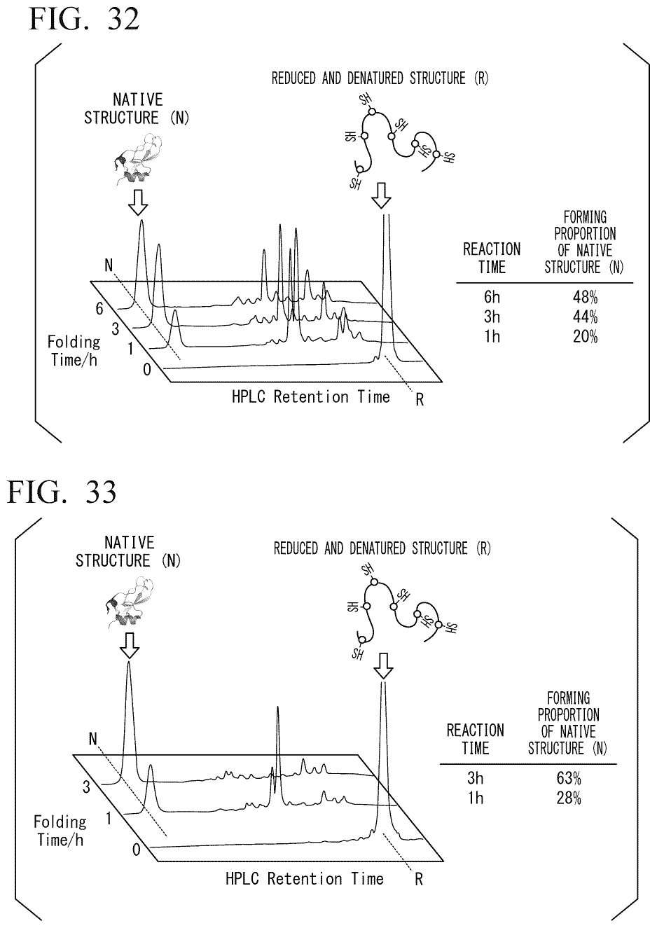

The results in the presence of Ni(II) ions are shown in FIG. 32. In the presence of Ni(II) ions, the protein folding promotion effect of the compound (B-2-1-1) was significantly improved as compared with the absence of Ni(II) ions (FIG. 31).

### (Protein Folding Test in Presence of Cu(II) Ion Using a Catalytic Amount of Compound (B-2-1-1))

In the presence of 30 μM of the compound (B-2-1-1), CuCl₂ (5 μM) and reduced and denatured BPTI (30 μM) were dissolved in a Tris-HCl buffer (50 mM Tris-HCl, 300 mM NaCl, pH: 7.5) and incubated at 30° C. The reaction solution (200 μL) after a certain period of time from the start of the reaction was mixed with an equivalent amount of 1 M hydrochloric acid and analyzed by reverse-phase HPLC. Tracing was performed at a flow rate of 1.0 mL·min⁻¹ and a wavelength of 229 nm, and a linear gradient was applied to the elution solvent ratio during measurement (solvent A: water containing 0.05% TFA, solvent B: acetonitrile containing 0.05% TFA; the amount of solvent A: 95% at 0 minutes, 80% at 15 minutes, 65% at 45 minutes).

The results are shown in FIG. 33. In the presence of Cu(II) ions, the protein folding promotion effect of the compound (B-2-1-1) was significantly improved as compared with the absence of Cu(II) ions (FIG. 31).

Since the native structural BPTI contains three disulfide bonds, three equivalents of an oxidizing agent, that is, the compound (B-2-1-1) is required for folding of BPTI. However, in the presence of Ni(II) ions or Cu(II) ions, the addition of a catalytic amount of the compound (B-2-1-1) resulted in enhancement of folding that greatly exceeded the theoretical yield expected for a stoichiometric reaction. Therefore, it is suggested that a complex formed between the compound (B-2-1-1) and the transition metal ion catalytically promoted the oxidative folding of BPTI.

### Experimental Example 4

### (Folding Test of Reduced and Denatured RNAase A in Presence of Cu(II) Ion Using Compound (A-1-1-1) and Compound (B-1-1-1))

RNase A (16 mg) was dissolved in 0.2 mM Tris-HCl (pH: 8.7, 0.5 mL) containing 6.0 M guanidine hydrochloride and 100 mM DTT, and incubated at 25° C. for 2 hours. Thereafter, dialysis was carried out with 10 mM hydrochloric acid for 2 hours to remove guanidine hydrochloride and DTT. The dialysis operation was repeated three times. As a result, a reduced and denatured RNAase A solution was obtained. The reduced and denatured RNAase A solution after the dialysis operation was stored at-30° C. until use in the folding experiment.

In the presence of 32 μM of oxidized glutathione (GSSG) and 320 μM of reduced glutathione (GSH), or in the presence of 320 μM of the compound (B-1-1-1) and 32 μM of the compound (A-1-1-1), 64 μM of CuCl₂ and reduced and denatured RNase A (8 μM) were dissolved in a Tris-HCl buffer (50 mM Tris-HCl, 300 mM NaCl, pH: 7.5) and incubated at 30° C. After a certain period of time from the start of the reaction, the reaction solution was mixed with a substrate oligonucleotide fluorescently labeled at both ends, and the enzyme activity of RNase A was quantified by measuring the change in fluorescence intensity upon cleavage of the substrate. As the substrate oligonucleotide, 5′-FAM-dA rU dA dA-TAMRA-3′ (dA: deoxyadenosine; rU: uridine), labeled with 6-carboxyfluorescein (FAM) and 5-carboxytetramethylrhodamine (TAMRA), was used. For the measurement of fluorescence intensity, a spectrofluorometer (RF-6000, Shimadzu Corporation) was used.

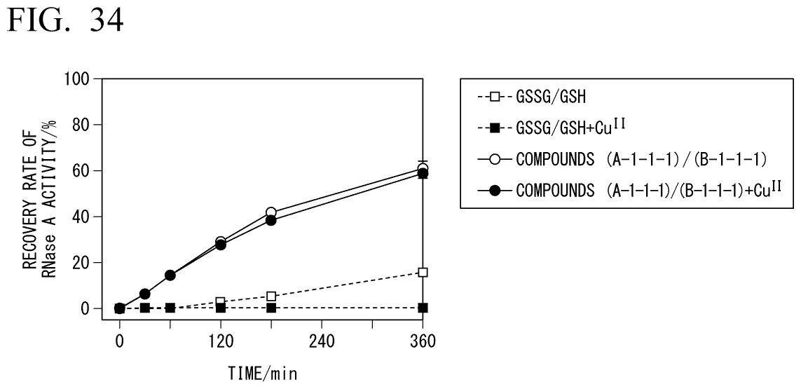

The results are shown in FIG. 34. When GSSG/GSH (oxidized glutathione and reduced glutathione), which are existing protein folding promoters, were used, the addition of Cu(II) ion significantly decreased the enzyme activity recovery rate of RNase A. Therefore, it was found that oxidative folding of RNase A into a native structure was inhibited in the presence of Cu(II) ions.

On the other hand, when the compound (A-1-1-1)/(B-1-1-1) was used, the enzyme activity recovery rate of RNase A was remarkably higher than that when GSH/GSSG was used, regardless of the presence or absence of Cu(II) ions.

From these results, it was found that the compounds (A-1-1-1) and (B-1-1-1) promoted oxidative folding of RNase A even in the coexistence of a transition metal ion, and the folding efficiency exceeded that of existing folding agents.

### INDUSTRIAL APPLICABILITY

According to the present invention, there are provided a protein folding agent capable of promoting protein folding in the presence of transition metal ions; a compound usable in the protein folding agent; a protein folding method using the protein folding agent; a pharmaceutical composition containing the protein folding agent; and an agent for suppressing cytotoxicity by a transition metal.

Hereinabove, preferable examples of the present invention have been described above, but the present invention is not limited to these examples. Configurations can be added, omitted, and replaced, and other modifications can be made within a range not departing from the gist of the present invention. The present invention is not limited by the above description, but only by the scope of the appended claims.

1. A protein folding method, comprising
incubating a protein in a presence of a protein folding agent comprising at least one or more compounds selected from the group consisting of the following (A) to (E):
(A) a cyclic polyamine that contains one or more thiol groups or disulfide bonds, is bindable to a transition metal ion, and is a compound represented by General Formula (A). 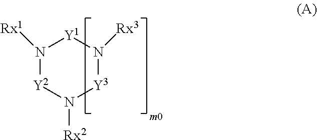
wherein Rx¹ to Rx³ each independently represent a hydrogen atom or a monovalent hydrocarbon group that may have a substituent that is a substituent which substitutes a hydrogen atom of the monovalent hydrocarbon group to a thiol group, a hydroxy group, an amino group, a halogen atom, an alkoxy group, or an acyl group, or that is a substituent which substitutes a methylene group of the monovalent hydrocarbon group to a ether bond, a carbonyl group, or an ester bond, provided that at least one of Rx¹ to Rx³ contains one or more thiol groups, when Rx¹ to Rx³ contain two or more thiol groups in total, the thiol groups may be bonded to each other to form a disulfide bond, Y¹ to Y³ each independently represent an alkylene group having 1 to 5 carbon atoms, m0 represents an integer of 1 to 3, when m0 is an integer of 2 or more, two or more Rx³'s may be the same as or different from each other, and two or more Y³'s may be the same as or different from each other;
(B) a compound in which two or more of the cyclic polyamines (A) are bonded through a disulfide bond between the thiol groups;
(C) a complex of the cyclic polyamine (A) or the compound (B) with a transition metal ion;
(D) a salt of the cyclic polyamine (A) or the compound (B); and
(E) a solvate of the cyclic polyamine (A) or the compound (B).

2. (canceled)

3. A protein folding method, comprising incubating a protein folding agent comprising at least one or more compounds selected from the group consisting of the following (A) to (E):
(A) a cyclic polyamine that contains one or more thiol groups or disulfide bonds, is bindable to a transition metal ion, and is a compound represented by General Formula (A-1) or (A-2), 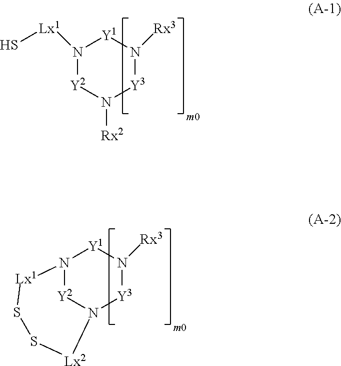
wherein, Rx² and Rx³ each independently represent a hydrogen atom or a monovalent hydrocarbon group that may have a substituent that is a substituent which substitutes a hydrogen atom of the monovalent hydrocarbon group to a thiol group, a hydroxy group, an amino group, a halogen atom, an alkoxy group, or an acyl group or that is a substituent which substitutes a methylene group of the monovalent hydrocarbon group to a ether bond, a carbonyl group, or an ester bond, Y¹ to Y³ each independently represent an alkylene group having 1 to 5 carbon atoms, Lx¹ and Lx² each independently represent an alkylene group having 1 to 10 carbon atoms, which may contain at least one selected from the group consisting of an oxygen atom and a nitrogen atom, m0 represents an integer of 1 to 3, and when m0 is an integer of 2 or more, two or more Rx³'s may be the same as or different from each other, and two or more Y³'s may be the same as or different from each other,
(B) a compound in which two or more of the cyclic polyamines (A) are bonded through a disulfide bond between the thiol groups;
(C) a complex of the cyclic polyamine (A) or the compound (B) with a transition metal ion;
(D) a salt of the cyclic polyamine (A) or the compound (B); and
(E) a solvate of the cyclic polyamine (A) or the compound (B).

4. The protein folding method according to claim 3, wherein the cyclic polyamine (A) is a compound represented by General Formula (A-1-1), and the compound (B) is a compound represented by General Formula (B-1-1), 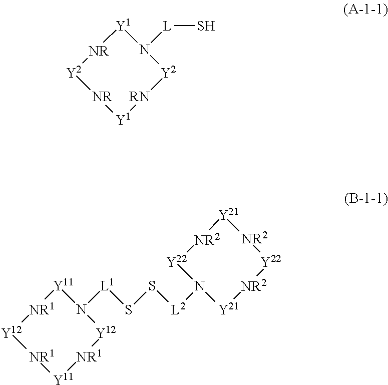
wherein, Y¹, Y², Y¹¹, Y¹², Y²¹, and Y²² each independently represent an alkylene group having 1 to 5 carbon atoms, R, R¹, and R² each independently represent a hydrogen atom or an alkyl group having 1 to 3 carbon atoms, and L, L¹, and L² each independently represent an alkylene group having 1 to 10 carbon atoms, which may contain at least one selected from the group consisting of an oxygen atom and a nitrogen atom.

5. The protein folding method according to claim 4, wherein the protein folding agent includes at least one compound selected from the group consisting of the compound (B), a metal complex of the compound (B), a salt of the compound (B), and a solvate of the compound (B).

6. The protein folding method according to claim 4, wherein the compound (B) is a compound represented by Formula (B-1-1-1),

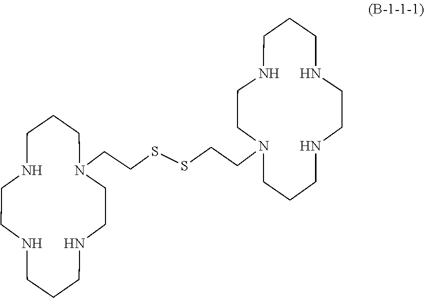 [SMILES: C1CNCCNCCCN(CCSSCCN2CCCNCCNCCCNCC2)CCNC1]

7. The protein folding method according to claim 4, wherein the cyclic polyamine (A) is a compound represented by Formula (A-1-1-1), and the compound (B) is a compound represented by Formula (B-1-1-1),

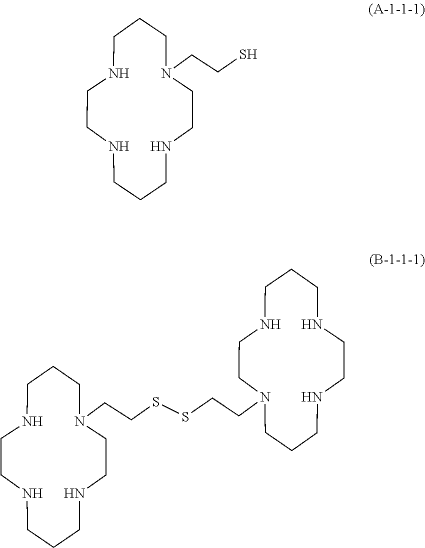 [SMILES: SCCN1CCCNCCNCCCNCC1 ; C1CNCCNCCCN(CCSSCCN2CCCNCCNCCCNCC2)CCNC1]

8. The protein folding method according to claim 3, wherein the cyclic polyamine (A) is a compound represented by General Formula (A-1-2), and the compound (B) is a compound represented by General Formula (B-1-2), 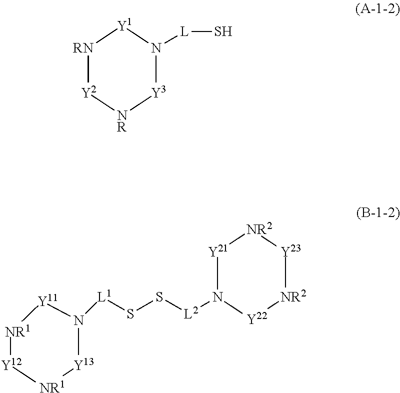
wherein Y¹, Y², Y³, Y¹¹, Y¹², Y¹³, Y²¹, Y²², and Y²³ each independently represent an alkylene group having 1 to 5 carbon atoms, R, R¹, and R² each independently represent a hydrogen atom or an alkyl group having 1 to 3 carbon atoms, and L, L¹, and L² each independently represent an alkylene group having 1 to 10 carbon atoms, which may contain at least one selected from the group consisting of an oxygen atom and a nitrogen atom.

9. The protein folding method according to claim 3, wherein the cyclic polyamine (A) is a compound represented by Formula (A-1-2-1), and the compound (B) is a compound represented by Formula (B-1-2-1),

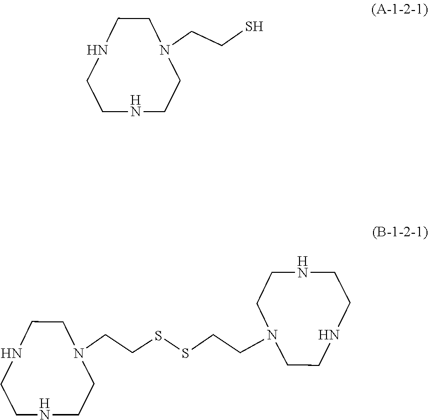 [SMILES: SCCN1CCNCCNCC1 ; C1CNCCN(CCSSCCN2CCNCCNCC2)CCN1]

10. The protein folding method according to claim 1,
wherein the protein folding agent includes at least one compound selected from the group consisting of a cyclic polyamine (A), a metal complex of the cyclic polyamine (A), a salt of the cyclic polyamine (A), and a solvate of the cyclic polyamine (A), and
at least one compound selected from the group consisting of the compound (B), a metal complex of the compound (B), a salt of the compound (B), and a solvate of the compound (B).

11. The protein folding method according to claim 3, wherein the cyclic polyamine (A) is a compound represented by Formula (A-2-1), 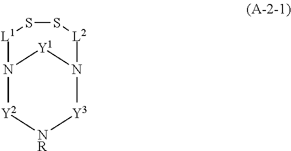
wherein, Y¹ to Y³ each independently represent an alkylene group having 1 to 5 carbon atoms, R represents a hydrogen atom or an alkyl group having 1 to 3 carbon atoms, and L¹ and L² each independently represent an alkylene group having 1 to 10 carbon atoms, which may contain at least one selected from the group consisting of an oxygen atom and a nitrogen atom.

12. The protein folding method according to claim 11, wherein the cyclic polyamine (A) is a compound represented by Formula (A-2-1-1),

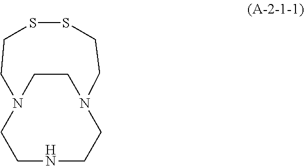 [SMILES: C1CN2CCSSCCN(CCN1)CC2]

13. The protein folding method according to claim 1, wherein the protein folding agent is used for folding a protein in a presence of a transition metal ion.

14. The protein folding method according to claim 13, wherein the protein is a protein containing a transition metal ion.

15. The protein folding method according to claim 13, wherein the transition metal ion is at least one selected from the group consisting of a copper ion, a nickel ion, an iron ion, a manganese ion, a zinc ion, and a molybdenum ion.

16. A compound represented by General Formula (B-1), (A-1-2), or (A-2), 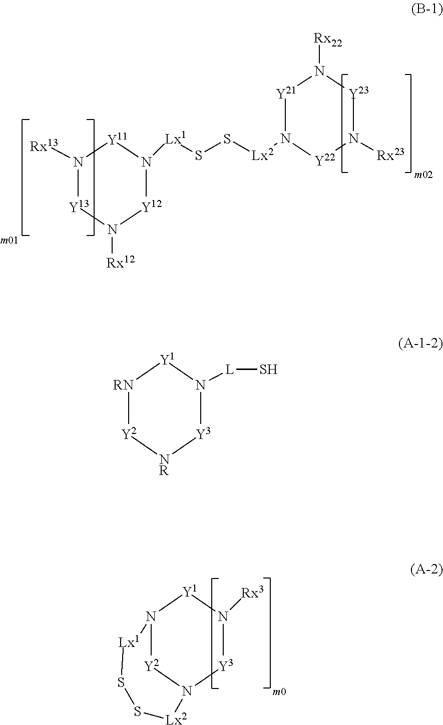
wherein, in General Formula (B-1), Rx¹², Rx¹³, Rx²², and Rx²³ each independently represent a hydrogen atom or a monovalent organic group, Y¹¹, Y¹², Y¹³, Y²¹, Y²², and Y²³ each independently represent an alkylene group having 1 to 5 carbon atoms, Lx¹ and Lx² each independently represent an alkylene group having 1 to 10 carbon atoms, which may contain at least one selected from the group consisting of an oxygen atom and a nitrogen atom, m01 and m02 each independently represent an integer of 1 to 3, when m01 is an integer of 2 or more, two or more Rx¹³'s may be the same as or different from each other, and two or more Y¹³'s may be the same as or different from each other, and when m02 is an integer of 2 or more, two or more Rx²³'s may be the same as or different from each other, and two or more Y²³'s may be the same as or different from each other;
in General Formula (A-1-2), Y¹ to Y³ each independently represent an alkylene group having 1 to 5 carbon atoms, R's each independently represent a hydrogen atom or an alkyl group having 1 to 3 carbon atoms, and L represents an alkylene group having 1 to 10 carbon atoms, which may contain at least one selected from the group consisting of an oxygen atom and a nitrogen atom;
in General Formula (A-2), Rx³ represents a hydrogen atom or a monovalent organic group, Y¹ to Y³ each independently represent an alkylene group having 1 to 5 carbon atoms, Lx¹ and Lx² each independently represent an alkylene group having 1 to carbon atoms, which may contain at least one selected from the group consisting of an oxygen atom and a nitrogen atom, m0 represents an integer of 1 to 3, and when m0 is an integer of 2 or more, two or more Rx³'s may be the same as or different from each other, and two or more Y³'s may be the same as or different from each other.

17. The compound according to claim 16, wherein the compound is a compound represented by Formula (B-1-1-1), (A-1-2-1), (B-1-2-1), or (A-2-1-1),

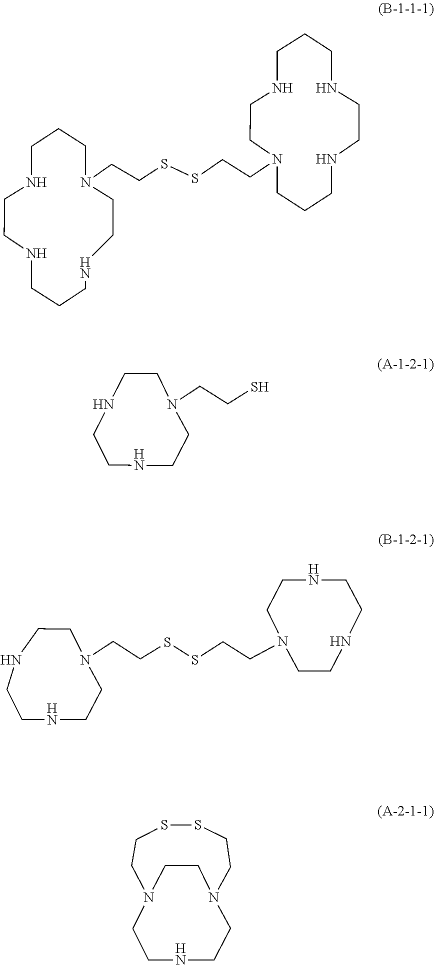 [SMILES: C1CNCCNCCCN(CCSSCCN2CCCNCCNCCCNCC2)CCNC1 ; SCCN1CCNCCNCC1 ; C1CNCCN(CCSSCCN2CCNCCNCC2)CCN1 ; C1CN2CCSSCCN(CCN1)CC2]

18. (canceled)

19. The protein folding method according to claim 1, wherein the protein is an unfolded protein or a misfolded protein.

20. The protein folding method according to claim 1, wherein the incubation is performed in a presence of a transition metal ion.

21. The protein folding method according to claim 20, wherein a folded protein after the incubating-step-incubation contains the transition metal ion.

22. The protein folding method according to claim 20, wherein the transition metal ion is at least one selected from the group consisting of a copper ion, a nickel ion, an iron ion, a manganese ion, a zinc ion, and a molybdenum ion.

23. A method for treating or preventing a disease caused by aggregation of proteins, comprising administering to a subject a protein folding agent comprising at least one or more compounds selected from the group consisting of the following (A) to (E):
(A) a cyclic polyamine that contains one or more thiol groups or disulfide bonds, is bindable to a transition metal ion, and is a compound represented by General Formula (A) 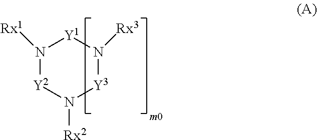
wherein Rx¹ to Rx³ each independently represent a hydrogen atom or a monovalent hydrocarbon group that may have a substituent that is a substituent which substitutes a hydrogen atom of the monovalent hydrocarbon group to a thiol group, a hydroxy group, an amino group, a halogen atom, an alkoxy group, or an acyl group, or that is a substituent which substitutes a methylene group of the monovalent hydrocarbon group to a ether bond, a carbonyl group, or an ester bond, provided that at least one of Rx¹ to Rx³ contains one or more thiol groups, when Rx¹ to Rx³ contain two or more thiol groups in total, the thiol groups may be bonded to each other to form a disulfide bond, Y¹ to Y³ each independently represent an alkylene group having 1 to 5 carbon atoms, m0 represents an integer of 1 to 3, when m0 is an integer of 2 or more, two or more Rx³'s may be the same as or different from each other, and two or more Y³'s may be the same as or different from each other:
(B) a compound in which two or more of the cyclic polyamines (A) are bonded through a disulfide bond between the thiol groups;
(C) a complex of the cyclic polyamine (A) or the compound (B) with a transition metal ion;
(D) a salt of the cyclic polyamine (A) or the compound (B); and
(E) a solvate of the cyclic polyamine (A) or the compound (B)

24. A method for treating or preventing a disease caused by aggregation of proteins, comprising administering to a subject a protein folding agent comprising at least one or more compounds selected from the group consisting of the following (A) to (E):
(A) a cyclic polyamine that contains one or more thiol groups or disulfide bonds, is bindable to a transition metal ion, and is a compound represented by General Formula (A-1) or (A-2), 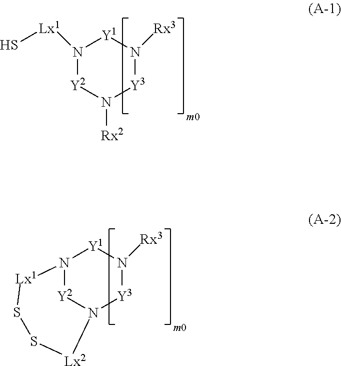
wherein, Rx² and Rx³ each independently represent a hydrogen atom or a monovalent hydrocarbon group that may have a substituent that is a substituent which substitutes a hydrogen atom of the monovalent hydrocarbon group to a thiol group, a hydroxy group, an amino group, a halogen atom, an alkoxy group, or an acyl group, or that is a substituent which substitutes a methylene group of the monovalent hydrocarbon group to a ether bond, a carbonyl group, or an ester bond, Y¹ to Y³ each independently represent an alkylene group having 1 to 5 carbon atoms, Lx¹ and Lx² each independently represent an alkylene group having 1 to 10 carbon atoms, which may contain at least one selected from the group consisting of an oxygen atom and a nitrogen atom, m0 represents an integer of 1 to 3, and when m0 is an integer of 2 or more, two or more Rx³'s may be the same as or different from each other, and two or more Y³'s may be the same as or different from each other;
(B) a compound in which two or more of the cyclic polyamines (A) are bonded through a disulfide bond between the thiol groups;
(C) a complex of the cyclic polyamine (A) or the compound (B) with a transition metal ion;
(D) a salt of the cyclic polyamine (A) or the compound (B); and
(E) a solvate of the cyclic polyamine (A) or the compound (B).

25. The method according to claim 24, wherein the disease caused by aggregation of proteins is a folding disease.

26. The method according to claim 25, wherein the folding disease is at least one selected from the group consisting of amyotrophic lateral sclerosis (ALS), Parkinson's disease, Alzheimer's disease, dementia with Lewy bodies, spinocerebellar degeneration, frontotemporal dementia, Pick's disease, Creutzfeldt-Jakob disease, familial amyloid polyneuropathy, and type 2 diabetes.

27. (canceled)
